(19) United States
(12) Patent Application Publication
(10) Pub. No.: US 2025/0189481 A1
(43) Pub. Date: Jun. 12, 2025

(54) GAS SENSOR AND METHOD FOR CONTROLLING GAS SENSOR

(71) Applicant: NGK INSULATORS, LTD., Nagoya-shi (JP)

(72) Inventors: Yusuke WATANABE, Nagoya-shi (JP); Daichi ICHIKAWA, Nagoya-shi (JP)

(21) Appl. No.: 19/060,949

(22) Filed: Feb. 24, 2025

Related U.S. Application Data
(63) Continuation of application No. PCT/JP2023/025907, filed on Jul. 13, 2023.

(30) Foreign Application Priority Data
Sep. 12, 2022 (JP) 2022-144890

(51) Int. Cl. G01N 27/41; G01N 33/00

(52) U.S. Cl. CPC G01N 27/41; G01N 33/0027

(57) ABSTRACT

A gas sensor includes a sensor element and a control unit. The sensor element includes: a base part; a measurement-object gas flow cavity; an oxygen pump cell; a decomposing pump cell including an intracavity decomposing pump electrode; an intracavity detection electrode; and a reference electrode. A pump control part of the control unit operates the decomposing pump cell so that a voltage between the intracavity detection electrode and the reference electrode is at a predetermined value, thereby decomposing at least a part of the target gas to be measured at the intracavity decomposing pump electrode and pumping out oxygen generated by decomposing the target gas to be measured. A concentration calculating part of the control unit calculates a concentration of the target gas to be measured in the measurement-object gas based on a value of a voltage between the intracavity decomposing pump electrode and the reference electrode.

### CROSS-REFERENCE TO RELATED APPLICATION

This application is a continuation application of PCT/JP2023/025907, filed on Jul. 13, 2023, which claims priority from Japanese Patent Application No. 2022-144890, filed on Sep. 12, 2022, the entire contents of which are incorporated herein by reference.

### BACKGROUND OF THE INVENTION

### Technical Field of the Invention

The present invention relates to a gas sensor and a control method of the gas sensor.

### Background Art

Measurement of concentration of an objective gas component (oxygen O₂, water vapor H₂O, carbon dioxide CO₂, nitrogen oxide NOx, ammonia NH₃, hydrocarbon HC, carbon dioxide CO₂, etc.) in a measurement-object gas is required in various fields, for example, in a combustion control and an exhaust gas control of an internal combustion engine such as an engine of automobile, in an environment control, in a medical field, a biotechnology field, and an agriculture and industry field. Various measurement devices are used for the concentration measurement, and as an example, a limiting current-type gas sensor using an oxygen ion conductive solid electrolyte such as zirconia (ZrO₂) is known.

For example, JP 5918177 B2 and JP 6469464 B2 disclose a gas sensor that identifies concentrations of a water vapor component and a carbon dioxide component of a measurement gas based on a current flowing through a solid electrolyte. JP 6469462 B2 also discloses a gas sensor that identifies a concentration of a water vapor component of a measurement gas based on a current flowing through the solid electrolyte.

Further, for example, JP 5323752 B2 discloses a NOx sensor that has a main pump cell and an auxiliary pump cell for adjusting an oxygen concentration, and a measurement pump cell including a measurement electrode for detecting NOx. In the NOx sensor, an oxygen partial pressure in a measurement-object gas is controlled to such a low level that does not substantially affect NOx measurement by the main pump cell and the auxiliary pump cell. NOx in the measurement-object gas whose oxygen partial pressure has been controlled is reduced in the measurement electrode, and a resulting oxygen is pumped out by the measurement pump cell to be detected as a current value.

### CITATION LIST

### Patent Documents

Patent Document 1: JP 5918177 B2

Patent Document 2: JP 6469464 B2

Patent Document 3: JP 6469462 B2

Patent Document 4: JP 5323752 B2

### SUMMARY OF THE INVENTION

### Problems to be solved by the Invention

With the tightening of automobile exhaust emission regulations, diversification of use environment, and the like, a gas sensor is required to accurately measure even a lower-concentration target gas to be measured. Here, a concentration range intended by the low concentration may vary depending on a kind of the target gas to be measured and the use environment of the gas sensor. When the target gas to be measured is water vapor H₂O or carbon dioxide CO₂, the low concentration means a concentration of, for example, about lower than 10%, lower than 5%, lower than 3%, or lower than 1%. When the target gas to be measured is nitrogen oxide NOx, ammonia NH₃, or the like, the low concentration means a concentration of, for example, about lower than 500 ppm, lower than 400 ppm, lower than 300 ppm, lower than 200 ppm, or lower than 100 ppm.

In a conventional limiting current type gas sensor, a concentration of a target gas to be measured is measured based on a current flowing through a solid electrolyte. For example, JP 5918177 B2 and JP 6469464 B2 disclose that substantially all of water vapor H₂O and carbon dioxide CO₂ as target gasses to be measured are decomposed in a main electrochemical pumping cell; hydrogen H₂ generated through the decomposition of water vapor H₂O is burned in a first measuring electrochemical pumping cell; and carbon monoxide CO generated through the decomposition of carbon dioxide CO₂ is burned in a second measuring electrochemical pumping cell. JP 5918177 B2 and JP 6469464 B2 also disclose that the gas sensor identifies each of the concentration of the water vapor component and the concentration of the carbon dioxide component based on the magnitude of each of currents flowing at those time. Further, for example, JP 5323752 B2 discloses that the oxygen generated by reduction of the target gas to be measured (for example, NOx) is detected as a current in the measurement pump cell. The magnitude of the current is to be a value corresponding to a concentration of the target gas to be measured.

By the way, even when the target gas to be measured is not present in the measurement-object gas, the current does not become at zero, and a small current flows through the first measuring electrochemical pumping cell and the second measuring electrochemical pumping cell, or the measurement pump cell (these are referred to as a measurement pump cell and the like). This small current is called an offset current. The offset current flows regardless of the concentration of the target gas to be measured. Thus, if the current value of the offset current changes for some factors, a current value detected in the measurement pump cell and the like shifts by the amount of the offset current value change regardless of the concentration of the target gas to be measured. When measuring low concentration of the target gas to be measured, the current value detected in the measuring pump cell and the like in accordance with the concentration of the target gas to be measured is relatively small, so the change in the current value due to the offset current value change is relatively large, and the effect on measurement accuracy tends to be large.

It is therefore an object of the present invention to accurately measure even a low-concentration target gas to be measured. Specifically, it is an object of the present invention to accurately measure a target gas to be measured in a wide concentration range including a low-concentration target gas to be measured.

### Means for solving the problems

As a result of intensive studies, the present inventors have found that, by configuring a gas sensor with:
an oxygen pump cell for adjusting an oxygen concentration in a measurement-object gas;
a decomposing pump cell including an intracavity decomposing pump electrode for decomposing at least a part of a target gas to be measured in the measurement-object gas whose oxygen concentration has been adjusted;
an intracavity detection electrode for detecting an oxygen concentration in the measurement-object gas in which the oxygen concentration has been adjusted and at least the part of the target gas to be measured has been decomposed; and
a reference electrode in contact with a reference gas, it is possible to accurately measure a target gas to be measured even when a concentration of the target gas to be measured is low.

The present invention is based on a new knowledge that a concentration of a target gas to be measured in a measurement-object gas can be calculated in accordance with a voltage between the intracavity decomposing pump electrode and the reference electrode when a voltage between the intracavity detection electrode and the reference electrode is set to a predetermined value.

The present invention is based on a knowledge that the decomposing pump cell including the intracavity decomposing pump electrode disposed upstream of the intracavity detection electrode is operated so that the voltage between the intracavity detection electrode and the reference electrode is at the predetermined value.

The present invention is based on a new knowledge that the decomposing pump cell including the intracavity decomposing pump electrode disposed upstream of the intracavity detection electrode is operated so that the voltage between the intracavity detection electrode and the reference electrode is at the predetermined value, and the concentration of the target gas to be measured in the measurement-object gas can be calculated in accordance with the voltage between the intracavity decomposing pump electrode and the reference electrode at that time.

The present invention includes the following aspects.

(1) A gas sensor for detecting a target gas to be measured in a measurement-object gas, the gas sensor comprising a sensor element and a control unit for controlling the sensor element, wherein
the sensor element comprises:
   a base part in an elongated plate shape, including an oxygen-ion-conductive solid electrolyte layer;
   a measurement-object gas flow cavity formed from one end part in a longitudinal direction of the base part;
   an oxygen pump cell for adjusting an oxygen concentration in a measurement-object gas, the oxygen pump cell including: an intracavity oxygen pump electrode disposed in the measurement-object gas flow cavity; and an extracavity oxygen pump electrode disposed at a position different from the measurement-object gas flow cavity on the base part and corresponding to the intracavity oxygen pump electrode;
   a decomposing pump cell for decomposing a target gas to be measured in the measurement-object gas, the decomposing pump cell including: an intracavity decomposing pump electrode disposed at a position farther from the one end part in the longitudinal direction of the base part than the intracavity oxygen pump electrode in the measurement-object gas flow cavity; and an extracavity decomposing pump electrode disposed at a position different from the measurement-object gas flow cavity on the base part and corresponding to the intracavity decomposing pump electrode;
   an intracavity detection electrode disposed at a position farther from the one end part in the longitudinal direction of the base part than the intracavity decomposing pump electrode in the measurement-object gas flow cavity; and
   a reference electrode disposed inside the base part to be in contact with a reference gas; and
the control unit comprises:
   a pump control part for controlling operation of the oxygen pump cell and the decomposing pump cell, and
   a concentration calculating part for calculating a concentration of the target gas to be measured in the measurement-object gas, wherein
the pump control part operates the oxygen pump cell to adjust an oxygen concentration in the measurement-object gas to a predetermined concentration,
the pump control part operates the decomposing pump cell so that a voltage between the intracavity detection electrode and the reference electrode is at a predetermined value, thereby decomposing at least a part of the target gas to be measured in the measurement-object gas at the intracavity decomposing pump electrode of the decomposing pump cell and pumping out, by the decomposing pump cell, oxygen generated by decomposing the target gas to be measured, and
the concentration calculating part calculates a concentration of the target gas to be measured in the measurement-object gas based on a value of a voltage between the intracavity decomposing pump electrode and the reference electrode.

(2) The gas sensor according to the above (1), wherein
the pump control part adjusts the voltage between the intracavity decomposing pump electrode and the reference electrode so that the voltage between the intracavity detection electrode and the reference electrode is at the predetermined value, thereby decomposing at least the part of the target gas to be measured in the measurement-object gas at the intracavity decomposing pump electrode of the decomposing pump cell and pumping out, by the decomposing pump cell, oxygen generated by decomposing the target gas to be measured, and
the concentration calculating part calculates the concentration of the target gas to be measured in the measurement-object gas based on a value of the adjusted voltage between the intracavity decomposing pump electrode and the reference electrode.

(3) The gas sensor according to the above (1) or (2), wherein
the sensor element further comprises a current measurement pump cell including the intracavity detection electrode, and an extracavity detection electrode disposed at a position different from the measurement-object gas flow cavity on the base part and corresponding to the intracavity detection electrode; and
the control unit further comprises a switching unit for switching whether a current flows through the current measurement pump cell or not.

(4) The gas sensor according to the above (3), wherein the control unit comprises:
a measurement mode switching part for switching between a voltage measurement mode in which a concentration of the target gas to be measured in the measurement-object gas is calculated based on the value of the voltage between the intracavity decomposing pump electrode and the reference electrode, and a current measurement mode in which a concentration of the target gas to be measured in the measurement-object gas is calculated based on a current value flowing through the current measurement pump cell, and
the measurement mode switching part switches the switching unit so that a current does not flow through the current measurement pump cell in case of switching to the voltage measurement mode, and switches the switching unit so that a current flows through the current measurement pump cell in case of switching to the current measurement mode.

(5) The gas sensor according to the above (4), wherein
the measurement mode switching part switches to the voltage measurement mode when the measurement mode switching part determines that a concentration of the target gas to be measured detected in the current measurement mode is lower than a predetermined first concentration threshold value C1, and
the measurement mode switching part switches to the current measurement mode when the measurement mode switching part determines that a concentration of the target gas to be measured detected in the voltage measurement mode is higher than a predetermined second concentration threshold value C2.

(6) The gas sensor according to the above (5), wherein the first concentration threshold value C1 is lower than the second concentration threshold value C2.

(7) The gas sensor according to any one of the above (4) to (6), wherein
in the current measurement mode, a current value in the current measurement pump cell is controlled so that a voltage between the intracavity detection electrode and the reference electrode is at a predetermined value.

(8) The gas sensor according to any one of the above (1) to (7), wherein the target gas to be measured is selected from the group consisting of water vapor H₂O, carbon dioxide CO₂, and nitrogen oxide NOx.

(9) A control method of a gas sensor for detecting a target gas to be measured in a measurement-object gas, the gas sensor comprising a sensor element and a control unit for controlling the sensor element, wherein
the sensor element comprises:
   a base part in an elongated plate shape, including an oxygen-ion-conductive solid electrolyte layer;
   a measurement-object gas flow cavity formed from one end part in a longitudinal direction of the base part;
   an oxygen pump cell for adjusting an oxygen concentration in a measurement-object gas, the oxygen pump cell including: an intracavity oxygen pump electrode disposed in the measurement-object gas flow cavity; and an extracavity oxygen pump electrode disposed at a position different from the measurement-object gas flow cavity on the base part and corresponding to the intracavity oxygen pump electrode;
   a decomposing pump cell for decomposing a target gas to be measured in the measurement-object gas, the decomposing pump cell including: an intracavity decomposing pump electrode disposed at a position farther from the one end part in the longitudinal direction of the base part than the intracavity oxygen pump electrode in the measurement-object gas flow cavity; and an extracavity decomposing pump electrode disposed at a position different from the measurement-object gas flow cavity on the base part and corresponding to the intracavity decomposing pump electrode;
   an intracavity detection electrode disposed at a position farther from the one end part in the longitudinal direction of the base part than the intracavity decomposing pump electrode in the measurement-object gas flow cavity; and
   a reference electrode disposed inside the base part to be in contact with a reference gas; and
the control unit comprises:
   a pump control part for controlling operation of the oxygen pump cell and the decomposing pump cell, and
   a concentration calculating part for calculating a concentration of the target gas to be measured in the measurement-object gas, and
the control method comprising:
   a pump controlling step of operating the oxygen pump cell by the pump control part to adjust an oxygen concentration in the measurement-object gas to a predetermined concentration, and
   operating the decomposing pump cell so that a voltage between the intracavity detection electrode and the reference electrode is at a predetermined value by the pump control part, thereby decomposing at least a part of the target gas to be measured in the measurement-object gas at the intracavity decomposing pump electrode of the decomposing pump cell and pumping out, by the decomposing pump cell, oxygen generated by decomposing the target gas to be measured, and
   a concentration calculating step of calculating a concentration of the target gas to be measured in the measurement-object gas based on a value of a voltage between the intracavity decomposing pump electrode and the reference electrode by the concentration calculating part.

(10) The control method according to the above (9), wherein
the sensor element further comprises a current measurement pump cell including the intracavity detection electrode, and an extracavity detection electrode disposed at a position different from the measurement-object gas flow cavity on the base part and corresponding to the intracavity detection electrode; and
the control unit further comprises a switching unit for switching whether a current flows through the current measurement pump cell or not, and
the control method comprises:
   a step of performing a voltage measurement mode in which the pump control step and the concentration calculating step are performed to calculate a concentration of the target gas to be measured in the measurement-object gas based on the value of the voltage between the intracavity decomposing pump electrode and the reference electrode, or a current measurement mode in which the oxygen pump cell, the decomposing pump cell and the current measurement pump cell are operated to calculate a concentration of the target gas to be measured in the measurement-object gas based on a current value in the current measurement pump cell, while switching between the voltage measurement mode and the current measurement mode by using the switching unit.

### Advantageous Effect of the Invention

According to the present invention, it is possible to accurately measure even a low-concentration target gas to be measured. Specifically, according to the present invention, it is possible to accurately measure a target gas to be measured in a wide concentration range including a low-concentration target gas to be measured.

### BRIEF DESCRIPTION OF THE DRAWINGS

FIG. 1 is a vertical sectional schematic view in a longitudinal direction, showing one example of a general configuration of a gas sensor 100.

FIG. 2 is a block diagram showing electric connections between a control unit 90 and respective pump cells 21 and 50, respective sensor cells 80, 81, 82, and 83, and heater part 70 of a sensor element 101.

FIG. 3 is a conceptual diagram showing a principle of measuring water vapor in the gas sensor 100.

FIG. 4 is a graph showing one example of a relationship between a voltage V1 and a voltage V2 in the gas sensor 100. The horizontal axis of the graph represents the voltage V1 (mV) and the vertical axis of the graph represents the voltage V2 (mV).

FIG. 5 is a graph showing one example of a relationship between H₂O concentration and the voltage V1 in the gas sensor 100. The horizontal axis of the graph represents the H₂O concentration (%) and the vertical axis of the graph represents the voltage V1 (mV) when the voltage V2 is at 450 mV.

FIG. 6 is a vertical sectional schematic view in a longitudinal direction, showing one example of a general configuration of a gas sensor 200 of a variation.

FIG. 7 is a block diagram showing electric connections between a control unit 290 and respective pump cells 21, 50, and 41, respective sensor cells 80, 81, 82, and 83, and heater part 70 of a sensor element 201, in the gas sensor 200 of the variation.

FIG. 8 is a graph schematically showing one example of time variation of H₂O concentration detected value output by the gas sensor 200 of the variation and switching of measurement modes. The horizontal axis of the graph represents time (seconds) and the vertical axis of the graph represents the H₂O concentration detected value (%) output by the gas sensor 200.

FIG. 9 is a flow chart showing one example of detecting process of H₂O concentration in the gas sensor 200 of the variation.

FIG. 10 is a graph showing one example of a relationship between CO₂ concentration and the voltage V1 in a gas sensor of another embodiment. The horizontal axis of the graph represents the CO₂ concentration (%) and the vertical axis of the graph represents the voltage V1 (mV) when the voltage V2 is at 450 mV.

FIG. 11 is a conceptual diagram showing a principle of measuring nitrogen oxide, in the case of measuring nitrogen oxide by using the gas sensor 100.

### MODES FOR CARRYING OUT OF THE INVENTION

A gas sensor of the present invention includes a sensor element and a control unit for controlling the sensor element.

The sensor element contained in the gas sensor of the present invention includes:
a base part in an elongated plate shape, including an oxygen-ion-conductive solid electrolyte layer;
a measurement-object gas flow cavity formed from one end part in a longitudinal direction of the base part;
an oxygen pump cell for adjusting an oxygen concentration in a measurement-object gas, the oxygen pump cell including: an intracavity oxygen pump electrode disposed in the measurement-object gas flow cavity; and an extracavity oxygen pump electrode disposed at a position different from the measurement-object gas flow cavity on the base part and corresponding to the intracavity oxygen pump electrode;
a decomposing pump cell for decomposing a target gas to be measured in the measurement-object gas, the decomposing pump cell including: an intracavity decomposing pump electrode disposed at a position farther from the one end part in the longitudinal direction of the base part than the intracavity oxygen pump electrode in the measurement-object gas flow cavity; and an extracavity decomposing pump electrode disposed at a position different from the measurement-object gas flow cavity on the base part and corresponding to the intracavity decomposing pump electrode;
an intracavity detection electrode disposed at a position farther from the one end part in the longitudinal direction of the base part than the intracavity decomposing pump electrode in the measurement-object gas flow cavity; and
a reference electrode disposed inside the base part to be in contact with a reference gas.

The control unit contained in the gas sensor of the present invention includes
a pump control part for controlling operation of the oxygen pump cell and the decomposing pump cell, and
a concentration calculating part for calculating a concentration of the target gas to be measured in the measurement-object gas, wherein
the pump control part operates the oxygen pump cell to adjust an oxygen concentration in the measurement-object gas to a predetermined concentration,
the pump control part operates the decomposing pump cell so that a voltage between the intracavity detection electrode and the reference electrode is at a predetermined value, thereby decomposing at least a part of the target gas to be measured in the measurement-object gas at the intracavity decomposing pump electrode of the decomposing pump cell and pumping out, by the decomposing pump cell, oxygen generated by decomposing the target gas to be measured, and
the concentration calculating part calculates a concentration of the target gas to be measured in the measurement-object gas based on a value of a voltage between the intracavity decomposing pump electrode and the reference electrode.

Hereinafter, an example of an embodiment of a gas sensor of the present invention will be described in detail.

### General Configuration of Gas Sensor

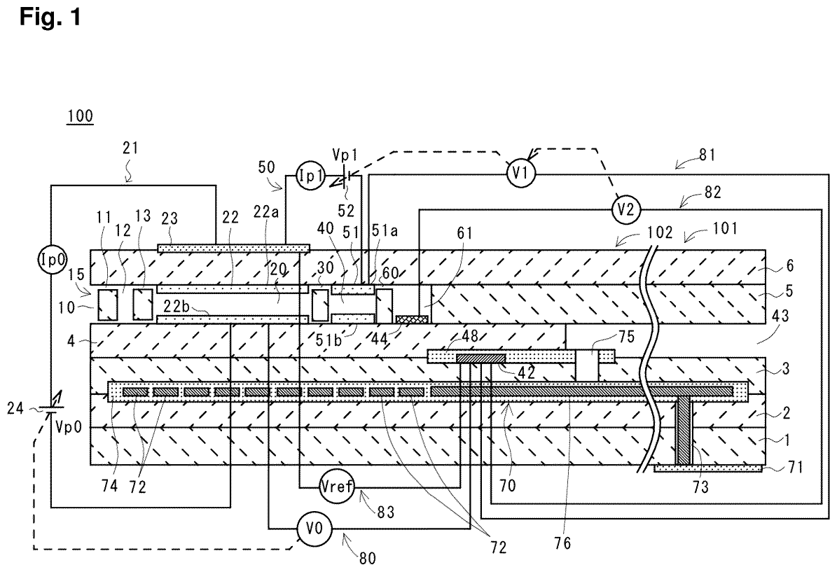

The gas sensor of the present invention will now be described with reference to the drawings. FIG. 1 is a vertical sectional schematic view in the longitudinal direction, showing one example of a general configuration of a gas sensor 100 including a sensor element 101. Hereinafter, based on FIG. 1, the upper side and the lower side in FIG. 1 are respectively defined as top and bottom, and the left side and the right side in FIG. 1 are respectively defined as a front end side and a rear end side.

In FIG. 1, the gas sensor 100 represents one example of a water vapor sensor (or, a H₂O sensor) that detects water vapor H₂O in a measurement-object gas by the sensor element 101, and measures the concentration of H₂O.

### (Sensor Element)

The sensor element 101 is an element in an elongated plate shape, including a base part 102 having such a structure that a plurality of oxygen-ion-conductive solid electrolyte layers are layered. The elongated plate shape also called a long plate shape or a belt shape. The base part 102 has such a structure that six layers, namely, a first substrate layer 1, a second substrate layer 2, a third substrate layer 3, a first solid electrolyte layer 4, a spacer layer 5, and a second solid electrolyte layer 6, are layered in this order from the bottom side, as viewed in the drawing. Each of the six layers is formed of an oxygen-ion-conductive solid electrolyte layer containing, for example, zirconia (ZrO₂). The solid electrolyte forming these six layers is dense and gastight. These six layers all may have the same thickness, or the thickness may vary among the layers. The layers are adhered to each other with an adhesive layer of a solid electrolyte interposed therebetween, and the base part 102 includes the adhesive layer. While a layer configuration composed of the six layers is illustrated in FIG. 1, the layer configuration in the present invention is not limited to this, and any number of layers and any layer configuration are possible.

The sensor element 101 is manufactured, for example, by stacking ceramic green sheets corresponding to the individual layers after conducting predetermined processing, printing of circuit pattern and the like, and then firing the stacked ceramic green sheets so that they are combined together.

A gas inlet 10 is formed between the lower surface of the second solid electrolyte layer 6 and the upper surface of the first solid electrolyte layer 4 in one end part in the longitudinal direction (hereinafter, referred to as a front end part) of the sensor element 101. A measurement-object gas flow cavity 15, that is, a measurement-object gas flow part is formed in such a form that a first diffusion-rate limiting part 11, a buffer space 12, a second diffusion-rate limiting part 13, a first internal cavity 20, a third diffusion-rate limiting part 30, a second internal cavity 40, a fourth diffusion-rate limiting part 60, and a third internal cavity 61 communicate in this order in the longitudinal direction from the gas inlet 10.

The gas inlet 10, the buffer space 12, the first internal cavity 20, the second internal cavity 40, and the third internal cavity 61 constitute internal spaces of the sensor element 101. Each of the internal spaces is provided in such a manner that a portion of the spacer layer 5 is hollowed out, and the top of each of the internal spaces is defined by the lower surface of the second solid electrolyte layer 6, the bottom of each of the internal spaces is defined by the upper surface of the first solid electrolyte layer 4, and the lateral surface of each of the internal spaces is defined by the lateral surface of the spacer layer 5.

Each of the first diffusion-rate limiting part 11, the second diffusion-rate limiting part 13, and the third diffusion-rate limiting part 30 is provided as two laterally elongated slits (having the longitudinal direction of the openings in the direction perpendicular to the figure in FIG. 1). Each of the first diffusion-rate limiting part 11, and the second diffusion-rate limiting part 13 may be in such a form that a desired diffusion resistance is created, but the form is not limited to the slits.

The fourth diffusion-rate limiting part 60 is provided as a single laterally elongated slit (having the longitudinal direction of the opening in the direction perpendicular to the figure in FIG. 1) between the spacer layer 5 and the second solid electrolyte layer 6. The fourth diffusion-rate limiting part 60 may be in such a form that a desired diffusion resistance is created, but the form is not limited to the slit.

Also, at a position farther from the front end than the measurement-object gas flow cavity 15, a reference gas introduction space 43 is disposed between the upper surface of the third substrate layer 3 and the lower surface of the spacer layer 5 at a position where the reference gas introduction space 43 is laterally defined by the lateral surface of the first solid electrolyte layer 4. The reference gas introduction space 43 has an opening in the other end part (hereinafter, referred to as a rear end part) of the sensor element 101. As a reference gas for H₂O concentration measurement, for example, air is introduced into the reference gas introduction space 43.

An air introduction layer 48 is a layer formed of porous alumina, and is so configured that a reference gas is introduced into the air introduction layer 48 via the reference gas introduction space 43. The air introduction layer 48 is formed to cover a reference electrode 42.

The reference electrode 42 is an electrode sandwiched between the upper surface of the third substrate layer 3 and the first solid electrolyte layer 4, and as described above, the air introduction layer 48 leading to the reference gas introduction space 43 is disposed around the reference electrode 42. That is, the reference electrode 42 is disposed to be in contact with a reference gas via the air introduction layer 48 which is a porous material, and the reference gas introduction space 43. As will be described later, the reference electrode 42 can be used to measure the oxygen concentration (oxygen partial pressure) in the first internal cavity 20, the second internal cavity 40, and the third internal cavity 61. The reference electrode 42 may be formed as a porous cermet electrode (e.g., a cermet electrode of Pt and ZrO₂).

In the measurement-object gas flow cavity 15, the gas inlet 10 is open to the external space, and the measurement-object gas is taken into the sensor element 101 from the external space through the gas inlet 10.

In the present embodiment, the measurement-object gas flow cavity 15 is in such a form that the measurement-object gas is introduced through the gas inlet 10 that is open on the front end surface of the sensor element 101, however, the present invention is not limited to this form. For example, the measurement-object gas flow cavity 15 need not have a recess of the gas inlet 10. In this case, the first diffusion-rate limiting part 11 substantially serves as a gas inlet.

For example, the measurement-object gas flow cavity 15 may have an opening that communicates with the buffer space 12 or a position near the buffer space 12 of the first internal cavity 20, on a lateral surface along the longitudinal direction of the base part 102. In this case, the measurement-object gas is introduced from the lateral surface along the longitudinal direction of the base part 102 through the opening.

Further, for example, the measurement-object gas flow cavity 15 may be so configured that the measurement-object gas is introduced through a porous body.

The first diffusion-rate limiting part 11 creates a predetermined diffusion resistance to the measurement-object gas taken through the gas inlet 10.

The buffer space 12 is provided to guide the measurement-object gas introduced from the first diffusion-rate limiting part 11 to the second diffusion-rate limiting part 13.

The second diffusion-rate limiting part 13 creates a predetermined diffusion resistance to the measurement-object gas introduced into the first internal cavity 20 from the buffer space 12.

It suffices that the amount of the measurement-object gas to be introduced into the first internal cavity 20 finally falls within a predetermined range. That is, it suffices that a predetermined diffusion resistance is created in a whole from the front end part of the sensor element 101 to the second diffusion-rate limiting part 13. For example, the first diffusion-rate limiting part 11 may directly communicate with the first internal cavity 20, or the buffer space 12 and the second diffusion-rate limiting part 13 may be absent.

The buffer space 12 is provided to mitigate the influence of pressure fluctuation on the detected value when the pressure of the measurement-object gas fluctuates.

When the measurement-object gas is introduced from outside the sensor element 101 into the first internal cavity 20, the measurement-object gas, which is rapidly taken through the gas inlet 10 into the sensor element 101 due to pressure fluctuation of the measurement-object gas in the external space (pulsations in exhaust pressure if the measurement-object gas is automotive exhaust gas), is not directly introduced into the first internal cavity 20. Rather, the measurement-object gas is introduced into the first internal cavity 20 after the pressure fluctuation of the measurement-object gas is eliminated through the first diffusion-rate limiting part 11, the buffer space 12, and the second diffusion-rate limiting part 13. Thus, the pressure fluctuation of the measurement-object gas introduced into the first internal cavity 20 becomes almost negligible.

The first internal cavity 20 is provided as a space for adjusting the oxygen partial pressure in the measurement-object gas introduced through the second diffusion-rate limiting part 13. The oxygen partial pressure is adjusted by operation of a main pump cell 21. That is, in the present embodiment, the main pump cell 21 functions as an oxygen pump cell for adjusting oxygen concentration in the measurement-object gas.

The main pump cell 21 is an electrochemical pump cell including the inner main pump electrode 22 as an intra-cavity oxygen pump electrode disposed on an inner surface of the measurement-object gas flow cavity 15, and the outer pump electrode 23 as an extra-cavity oxygen pump electrode disposed at a position different from the measurement-object gas flow cavity 15 on the base part 102 (in FIG. 1, on an outer surface of the base part 102) and corresponding to the inner main pump electrode 22. The phrase “corresponding to the inner main pump electrode 22” means that the outer pump electrode 23 and the inner main pump electrode 22 are provided with the second solid electrolyte layer 6 being interposed therebetween.

That is, the main pump cell 21 is an electrochemical pump cell composed of the inner main pump electrode 22 having a ceiling electrode portion 22a disposed over substantially the entire surface of the lower surface of the second solid electrolyte layer 6 that faces the first internal cavity 20, the outer pump electrode 23 disposed on a region of the upper surface of the second solid electrolyte layer 6 that corresponds to the ceiling electrode portion 22a so as to be exposed to the external space, and the second solid electrolyte layer 6 sandwiched between the inner main pump electrode 22 and the outer pump electrode 23.

The inner main pump electrode 22 is formed to span the upper and lower solid electrolyte layers (the second solid electrolyte layer 6 and the first solid electrolyte layer 4) that define the first internal cavity 20 and the spacer layer 5 that defines the lateral wall. Specifically, the ceiling electrode portion 22a is formed on the lower surface of the second solid electrolyte layer 6 that defines the ceiling surface of the first internal cavity 20, and a bottom electrode portion 22b is formed on the upper surface of the first solid electrolyte layer 4 that defines the bottom surface of the first internal cavity 20. Also, lateral electrode portions (not shown) are formed on the lateral wall surfaces (inner surface) of the spacer layer 5 that form both lateral wall parts of the first internal cavity 20 so as to connect the ceiling electrode portion 22a and the bottom electrode portion 22b. Thus, the inner main pump electrode 22 is provided as a tunnel-like structure in the area where the lateral electrode portions are disposed.

The inner main pump electrode 22 and the outer pump electrode 23 are porous cermet electrodes (electrodes in a state that a metal component and a ceramic component are mixed). The inner main pump electrode 22 and the outer pump electrode 23 may contain a noble metal having catalytic activity (e.g., at least one of Pt, Rh, Ir, Ru, and Pd) as the metal component. The ceramic component to be used is not particularly limited, but is preferably an oxygen-ion-conductive solid electrolyte as in the case of the base part 102. For example, each of the inner main pump electrode 22 and the outer pump electrode 23 may be formed as a porous cermet electrode made of Pt and ZrO₂.

In the main pump cell 21, a pump voltage Vp0 is applied between the inner main pump electrode 22 and the outer pump electrode 23 by a variable power supply 24 to make a pump current Ip0 flow between the inner main pump electrode 22 and the outer pump electrode 23 in either a positive or negative direction, and thus it is possible to pump out oxygen in the first internal cavity 20 to the external space or pump oxygen into the first internal cavity 20 from the external space.

The inner main pump electrode 22, the second solid electrolyte layer 6, the spacer layer 5, the first solid electrolyte layer 4, the third substrate layer 3, and the reference electrode 42 form an electrochemical sensor cell, namely, an oxygen-partial-pressure detection sensor cell 80 for main pump control. In the oxygen-partial-pressure detection sensor cell 80 for main pump control, an electromotive force (a voltage V0) is generated between the inner main pump electrode 22 and the reference electrode 42 due to a difference in oxygen concentration between an atmosphere in the first internal cavity 20 and an atmosphere in the reference gas introduction space 43.

The oxygen concentration (oxygen partial pressure) in the first internal cavity 20 can be detected from the voltage V0 measured in the oxygen-partial-pressure detection sensor cell 80 for main pump control. In addition, the pump current Ip0 is controlled by performing feedback control of the pump voltage Vp0 in the variable power supply 24 so that the voltage V0 is constant. Thus, the oxygen concentration in the first internal cavity 20 can be maintained at a predetermined constant value.

The third diffusion-rate limiting part 30 creates a predetermined diffusion resistance to the measurement-object gas whose oxygen concentration (oxygen partial pressure) has been controlled in the first internal cavity 20 by the operation of the main pump cell 21, and guides the measurement-object gas into the second internal cavity 40.

The second internal cavity 40 is provided as a space for reducing (decomposing) water vapor in the measurement-object gas introduced through the third diffusion-rate limiting part 30. The water vapor is reduced by operation of a decomposing pump cell 50.

The decomposing pump cell 50 is an electrochemical pump cell including a decomposing pump electrode 51 as an intracavity decomposing pump electrode disposed at a position farther from the front end portion in the longitudinal direction of the base part 102 than the inner main pump electrode 22 on the inner surface of the measurement-object gas flow cavity 15, and an extracavity decomposing pump electrode disposed at a position different from the measurement-object gas flow cavity 15 on the base part 102 and corresponding to the decomposing pump electrode 51. In this embodiment, the outer pump electrode 23 disposed on the outer surface of the base part 102 functions also as the extracavity decomposing pump electrode. The phrase “corresponding to the decomposing pump electrode 51” means that the outer pump electrode 23 and the decomposing pump electrode 51 are provided with the second solid electrolyte layer 6 being interposed therebetween.

That is, the decomposing pump cell 50 is an electrochemical pump cell composed of the decomposing pump electrode 51 having a ceiling electrode portion 51a disposed on substantially the entire surface of lower surface of the second solid electrolyte layer 6 facing with the second internal cavity 40, the outer pump electrode 23 (the outer electrode is not limited to the outer pump electrode 23, but may be any suitable electrode disposed at a position different from the measurement-object gas flow cavity 15), and the second solid electrolyte layer 6.

The decomposing pump electrode 51 is disposed in the second internal cavity 40 in a tunnel-like structure similar to the inner main pump electrode 22 disposed in the first internal cavity 20 described previously. Specifically, in the tunnel-like structure, the ceiling electrode portion 51a is formed on the second solid electrolyte layer 6 that defines the ceiling surface of the second internal cavity 40, a bottom electrode portion 51b is formed on the first solid electrolyte layer 4 that defines the bottom surface of the second internal cavity 40, and lateral electrode portions (not shown) connecting the ceiling electrode portion 51a and the bottom electrode portion 51b are formed on the wall surfaces of the spacer layer 5 that define the lateral walls of the second internal cavity 40.

The decomposing pump electrode 51 is an electrode having catalytic activity for decomposing water vapor. The decomposing pump electrode 51 is a porous cermet electrode, as with the case of the inner main pump electrode 22. The decomposing pump electrode 51 may contain a noble metal having catalytic activity (e.g., at least one of Pt, Rh, Ir, Ru, and Pd) as the metal component. The ceramic component to be used is not particularly limited, but is preferably an oxygen-ion-conductive solid electrolyte as in the case of the base part 102. For example, the decomposing pump electrode 51 may be a porous cermet electrode made of Pt and ZrO₂.

In the decomposing pump cell 50, by applying a voltage Vp1 between the decomposing pump electrode 51 and the outer pump electrode 23 by a variable power supply 52 to make a pump current Ip1 flow between the decomposing pump electrode 51 and the outer pump electrode 23, it is possible to decompose at least a part of water vapor at the decomposing pump electrode 51 (2H₂O→2H₂+O₂), and to pump out oxygen generated by the decomposition of water vapor from the second internal cavity 40 to the external space.

The decomposing pump electrode 51, the reference electrode 42, the second solid electrolyte layer 6, the spacer layer 5, the first solid electrolyte layer 4, and the third substrate layer 3 form an electrochemical sensor cell, namely, an oxygen-partial-pressure detection sensor cell 81 for decomposing pump control. In the oxygen-partial-pressure detection sensor cell 81 for decomposing pump control, an electromotive force (a voltage V1) is generated between the decomposing pump electrode 51 and the reference electrode 42 due to a difference in oxygen concentration between an atmosphere in the second internal cavity 40 and an atmosphere in the reference gas introduction space 43.

The oxygen concentration (oxygen partial pressure) in the second internal cavity 40 can be detected from the voltage V1 measured in the oxygen-partial-pressure detection sensor cell 81 for decomposing pump control. The pump voltage Vp1 in the variable power supply 52 is feedback controlled on the basis of the voltage V1 detected by the oxygen-partial-pressure detection sensor cell 81 for decomposing pump control.

The fourth diffusion-rate limiting part 60 creates a predetermined diffusion resistance to the measurement-object gas which contains hydrogen gas generated by decomposing at least a part of water vapor in the second internal cavity 40, and guides the measurement-object gas into the third internal cavity 61.

The third internal cavity 61 is provided as a space for measuring an oxygen partial pressure in the measurement-object gas introduced through the fourth diffusion-rate limiting part 60. The oxygen partial pressure is measured by an electromotive force detection sensor cell 82.

The electromotive force detection sensor cell 82 is an electrochemical sensor cell composed of a detection electrode 44 as an intracavity detection electrode, the reference electrode 42, the first solid electrolyte layer 4, and the third substrate layer 3. In the electromotive force detection sensor cell 82, an electromotive force (a voltage V2) is generated between the detection electrode 44 and the reference electrode 42 due to a difference in oxygen concentration between an atmosphere in the third internal cavity 61 and an atmosphere in the reference gas introduction space 43. It is to be noted that hydrogen gas is a reducing gas and therefore acts so as to increase the difference in the oxygen concentration.

The detection electrode 44 is an electrode disposed at a position farther from the one end part in the longitudinal direction of the base part 102 than the decomposing pump electrode 51 in the measurement-object gas flow cavity 15.

The detection electrode 44 is a porous cermet electrode disposed on the upper surface of the first solid electrolyte layer 4 in the third internal cavity 61. The detection electrode 44 may contain a noble metal having catalytic activity (e.g., at least one of Pt, Rh, Ir, Ru, and Pd) as the metal component. The ceramic component to be used is not particularly limited, but is preferably an oxygen-ion-conductive solid electrolyte as in the case of the base part 102. For example, the detection electrode 44 may be a porous cermet electrode made of Pt and ZrO₂.

The detection electrode 44 may further contain Au (gold). Alternatively, the detection electrode 44 may contain only Au as the metal component. It is considered that Au has catalytic activity with respect to hydrogen H₂, but is inactive with respect to carbon monoxide CO. When carbon dioxide CO₂ is present in the measurement-object gas, carbon dioxide may be decomposed at the decomposing pump electrode 51 to generate carbon monoxide CO. However, when the detection electrode 44 contains Au, it is considered that carbon monoxide CO is not detected in the detection electrode 44, and that only hydrogen H₂ generated by decomposing water vapor H₂O can be detected more accurately. The effect of carbon dioxide CO₂ can be decreased, and thus a water vapor concentration can be measured with higher accuracy. The metal component of the detection electrode 44 may appropriately be selected depending on a kind of the target gas to be measured.

The oxygen concentration (oxygen partial pressure) in the third internal cavity 61 can be detected from the electromotive force (the voltage V2) between the detection electrode 44 and the reference electrode 42 in the electromotive force detection sensor cell 82. The lower the concentration of oxygen gas, the larger the voltage V2. When hydrogen gas that is a reducing gas is present, the higher the concentration of hydrogen gas, the larger the voltage V2 tends to be. Feedback control is performed for adjusting a value of the voltage V1 between the decomposing pump electrode 51 and the reference electrode 42 in the oxygen-partial-pressure detection sensor cell 81 for decomposing pump control based on the voltage V2 detected in the electromotive force detection sensor cell 82.

Also, the second solid electrolyte layer 6, the spacer layer 5, the first solid electrolyte layer 4, the third substrate layer 3, the outer pump electrode 23, and the reference electrode 42 constitute an electrochemical sensor cell 83, and it is possible to detect the oxygen partial pressure in the measurement-object gas outside the sensor by an electromotive force Vref obtained by the sensor cell 83.

The sensor element 101 further includes a heater part 70 that functions as a temperature regulator of heating and maintaining the temperature of the sensor element 101 so as to enhance the oxygen ion conductivity of the solid electrolyte. The heater part 70 includes a heater electrode 71, a heater 72, a heater lead 76, a through hole 73, a heater insulating layer 74, and a pressure relief vent 75.

The heater electrode 71 is an electrode formed in contact with the lower surface of the first substrate layer 1. The power can be supplied to the heater part 70 from the outside by connecting the heater electrode 71 with a heater power supply 77 that is an external power supply.

The heater 72 is an electrical resistor sandwiched by the second substrate layer 2 and the third substrate layer 3 from top and bottom. The heater 72 is connected with the heater electrode 71 via a heater lead 76 that connects with the heater 72 and extends in the rear end side in the longitudinal direction of the sensor element 101, and the through hole 73. The heater 72 is externally powered by the heater power supply 77 through the heater electrode 71 to generate heat, and heats and maintains the temperature of the solid electrolyte forming the sensor element 101.

The heater 72 is embedded over the whole area from the first internal cavity 20 to the third internal cavity 61 so that the temperature of the entire sensor element 101 can be adjusted to such a temperature that activates the solid electrolyte. The temperature may be adjusted so that the main pump cell 21 and the decomposing pump cell 50 are operable and each of electromotive forces are detectable in the oxygen-partial-pressure detection sensor cell 80 for main pump control, the oxygen-partial-pressure detection sensor cell 81 for decomposing pump control, the electromotive force detection sensor cell 82, and the sensor cell 83. It is not necessary that the whole area is adjusted to the same temperature, but the sensor element 101 may have temperature distribution.

In the sensor element 101 of the present embodiment, the heater 72 is embedded in the base part 102, but this form is not limitative. The heater 72 may be disposed to heat the base part 102. That is, the heater 72 may heat the sensor element 101 to develop oxygen ion conductivity with which the main pump cell 21 and the decomposing pump cell 50 are operable. For example, the heater 72 may be embedded in the base part 102 as in the present embodiment. Alternatively, for example, the heater part 70 may be formed as a heater substrate that is separate from the base part 102, and may be disposed at a position adjacent to the base part 102.

The heater insulating layer 74 is formed of an insulator such as alumina on the upper and lower surfaces of the heater 72 and the heater lead 76. The heater insulating layer 74 is formed to ensure electrical insulation between the second substrate layer 2, and the heater 72 and the heater lead 76, and electrical insulation between the third substrate layer 3, and the heater 72 and the heater lead 76.

The pressure relief vent 75 extends through the third substrate layer 3 so that the heater insulating layer 74 and the reference gas introduction space 43 communicate with each other. The pressure relief vent 75 can mitigate an increase in internal pressure due to temperature rise in the heater insulating layer 74. The pressure relief vent 75 may be absent.

The above-described sensor element 101 is incorporated into the gas sensor 100 in such a form that the front end part of the sensor element 101 comes into contact with the measurement-object gas, and the rear end part of the sensor element 101 comes into contact with the reference gas.

### (Control Unit)

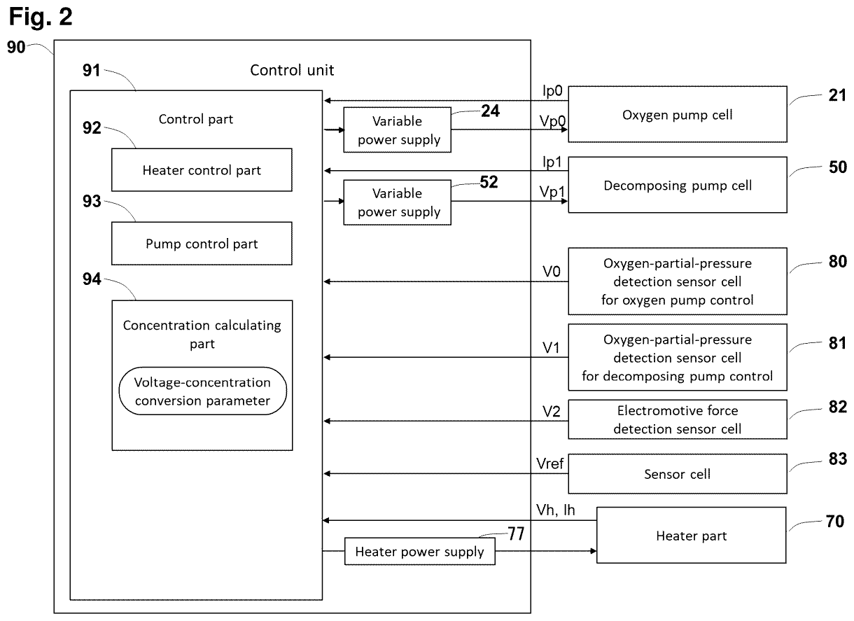

The gas sensor 100 of this embodiment includes the sensor element 101 described above and the control unit 90 for controlling the sensor element 101. In the gas sensor 100, each of the electrodes 22, 23, 51, 44, and 42 of the sensor element 101 is electrically connected to the control unit 90 through a lead wire not shown. FIG. 2 is a block diagram showing electric connections between the control unit 90 and the respective pump cells 21 and 50, the respective sensor cells 80, 81, 82, and 83, and the heater part 70 of the sensor element 101. The control unit 90 includes the above-described variable power supplies 24 and 52, the heater power supply 77, and a control part 91. The control part 91 includes a heater control part 92, a pump control part 93, and a concentration calculating part 94.

The control part 91 is realized by a general-purpose or dedicated computer, and functions as the heater control part 92, the pump control part 93, and the concentration calculating part 94 are realized by a CPU, a memory or the like installed in the computer. It is to be noted that when water vapor H₂O contained in exhaust gas from the engine of a car is a target gas to be measured by the gas sensor 100 and the sensor element 101 is attached to an exhaust gas path, some or all of the functions of the control unit 90 (especially, the control part 91) may be realized by an electronic control unit (ECU) installed in the car.

The control part 91 is configured to acquire an electromotive force (V0, V1, V2, Vref) in each of the sensor cells 80, 81, 82, and 83, a pump current (Ip0, Ip1) in each of the pump cells 21 and 50, and a heater voltage Vh and a heater current Ih in the heater part 70 of the sensor element 101. Further, the control part 91 is configured to output control signals to the variable power supplies 24 and 52, and the heater power supply 77.

The heater control part 92 may be configured to control the heater part 70 (especially, the heater 72). The heater control part 92 heats the heater 72, and maintains the temperature of the heater 72 at a desired temperature.

In order to heat the heater 72, known various control methods can be used. For example, the heater 72 may be heated by applying a certain voltage to the heater 72. The output of the heater power supply 77 may be controlled on the basis of the resistance value of the heater 72. Alternatively, the output of the heater power supply 77 may be controlled on the basis of at least one of resistance values in the main pump cell 21 and the decomposing pump cell 50. The output of the heater power supply 77 may also be controlled on the basis of at least one of resistance values in the oxygen-partial-pressure detection sensor cell 80 for main pump control, the oxygen-partial-pressure detection sensor cell 81 for decomposing pump control, the electromotive force detection sensor cell 82, and the sensor cell 83.

For example, the heater control part 92 performs feedback control of a control signal output to the heater power supply 77 on the basis of a heater resistance value Rh(=Vh/Ih) calculated from the heater voltage Vh and the heater current Ih in the heater 72 so that the heater 72 reaches a target temperature.

The pump control part 93 is configured to control the main pump cell 21 and the decomposing pump cell 50 so that the gas sensor 100 can measure a concentration of a target gas to be measured (in this embodiment, water vapor H₂O).

The pump control part 93 is configured to adjust an oxygen concentration in a measurement-object gas to a predetermined concentration by the oxygen pump cell (namely, the main pump cell 21). In this embodiment, the pump control part 93 performs feedback control of the pump voltage Vp0 of the variable power supply 24 in the main pump cell 21 so that the voltage V0 in the oxygen-partial-pressure detection sensor cell 80 for main pump control is at a constant value (referred to as a set value V0_{SET}). The set value V0_{SET} may be set, for example, so that oxygen partial pressure in the atmosphere in the first internal cavity 20 is controlled to be a low partial pressure that does not substantially affect measurement of H₂O. For example, the set value V0_{SET} may be set so that the oxygen partial pressure is controlled to be an oxygen partial pressure at which H₂O as the target gas to be measured is not substantially decomposed. The voltage V0 indicates the oxygen partial pressure in the vicinity of the inner main pump electrode 22, and therefore making the voltage V0 constant means that the oxygen partial pressure in the vicinity of the inner main pump electrode 22 is made constant. As a result, the pump current Ip0 in the main pump cell 21 varies depending on the oxygen concentration in the measurement-object gas.

When the oxygen partial pressure in the measurement-object gas is higher than the oxygen partial pressure corresponding to the set value V0_{SET}, the main pump cell 21 pumps oxygen out from the first internal cavity 20. On the other hand, when the oxygen partial pressure in the measurement-object gas is lower than the oxygen partial pressure corresponding to the set value V0_{SET} (for example, when hydrocarbons HC or the like are contained), the main pump cell 21 pumps oxygen into the first internal cavity 20 from the space outside the sensor element 101. Therefore, the value of the pump current Ip0 may be either positive or negative.

The pump control part 93 is configured to operate the decomposing pump cell 50 so that the voltage V2 between the intracavity detection electrode (namely, the detection electrode 44) and the reference electrode 42 in the electromotive force detection sensor cell 82 is at a predetermined value, thereby decomposing at least a part of the target gas to be measured in the measurement-object gas at the intracavity decomposing pump electrode (namely, the decomposing pump electrode 51) of the decomposing pump cell 50 and pumping out, by the decomposing pump cell 50, oxygen generated by decomposing the target gas to be measured.

The pump control part 93 may be configured to adjust the voltage V1 between the intracavity decomposing pump electrode (namely, the decomposing pump electrode 51) and the reference electrode 42 in the oxygen-partial-pressure detection sensor cell 81 for decomposing pump control so that the voltage V2 between the intracavity detection electrode (namely, the detection electrode 44) and the reference electrode 42 in the electromotive force detection sensor cell 82 is at the predetermined value, thereby decomposing at least the part of the target gas to be measured in the measurement-object gas at the intracavity decomposing pump electrode (namely, the decomposing pump electrode 51) of the decomposing pump cell 50 and pumping out, by the decomposing pump cell 50, oxygen generated by decomposing the target gas to be measured.

In this embodiment, the pump control part 93 performs feedback control to adjust a target value (referred to as a set value V1_{SET}) of the voltage V1 in the oxygen-partial-pressure detection sensor cell 81 for decomposing pump control so that the voltage V2 between the detection electrode 44 and the reference electrode 42 in the electromotive force detection sensor cell 82 is at a target value (referred to as a set value V2_{SET}). The pump control part 93 also performs feedback control of the pump voltage Vp1 of the variable power supply 52 in the decomposing pump cell 50 so that the voltage V1 in the oxygen-partial-pressure detection sensor cell 81 for decomposing pump control is at the target value (namely, the set value V1_{SET}) that has been adjusted. The set value V2_{SET} may appropriately be set, for example, depending on a kind of the target gas to be measured and a concentration range to be measured by the gas sensor 100. The set value V2_{SET} may vary depending on a target gas to be measured, but may be, for example, about 300 mV to 600 mV when water vapor is measured. More preferably, the set value V2_{SET} may be about 400 mV to 500 mV. The set value V2_{SET} may be, for example, 450 mV.

The concentration calculating part 94 is configured to calculate a concentration of a target gas to be measured (in this embodiment, a H₂O concentration) in a measurement-object gas.

The concentration calculating part 94 acquires the voltage V1 between the intracavity decomposing pump electrode (the decomposing pump electrode 51) and the reference electrode 42 in the oxygen-partial-pressure detection sensor cell 81 for decomposing pump control, and calculates the H₂O concentration in the measurement-object gas on the basis of a previously-stored conversion parameter (voltage-concentration conversion parameter) between the voltage V1 and the H₂O concentration in the measurement-object gas. And, the concentration calculating part 94 outputs the calculated H₂O concentration as a measurement value of the gas sensor 100. The voltage-concentration conversion parameter is previously stored, as data showing the relationship between the voltage V1 and the H₂O concentration in the measurement-object gas that will be described later, in the memory of the control part 91 which functions as the concentration calculating part 94. The voltage-concentration conversion parameter may appropriately be determined by those skilled in the art by, for example, previously performing an experiment on the gas sensor 100. The voltage-concentration conversion parameter may be, for example, the coefficient of an approximate expression (e.g., logarithmic function) obtained by experiment or a map showing the relationship between the voltage V1 and the H₂O concentration in the measurement-object gas. The voltage-concentration conversion parameter may be specific to each individual gas sensor 100 or may be common to a plurality of gas sensors.

The concentration calculating part 94 may further be configured to calculate an oxygen concentration based on the pump current Ip0 flowing through the main pump cell 21. This configuration makes it possible to simultaneously measure both of a concentration of the target gas to be measured (in this embodiment, a H₂O concentration) in the measurement-object gas, and an oxygen concentration in the measurement-object gas, by using the gas sensor 100.

### (Detection of Concentration of Target Gas to be Measured)

A method for measuring a water vapor concentration (a H₂O concentration) in the measurement-object gas by using the gas sensor 100 having such a configuration as described above will be described.

A control method of a gas sensor of the present invention includes:
a pump controlling step of operating the oxygen pump cell by the pump control part to adjust an oxygen concentration in the measurement-object gas to a predetermined concentration, and
operating the decomposing pump cell so that a voltage between the intracavity detection electrode and the reference electrode is at a predetermined value by the pump control part, thereby decomposing at least a part of the target gas to be measured in the measurement-object gas at the intracavity decomposing pump electrode of the decomposing pump cell and pumping out, by the decomposing pump cell, oxygen generated by decomposing the target gas to be measured, and
a concentration calculating step of calculating a concentration of the target gas to be measured in the measurement-object gas based on a value of a voltage between the intracavity decomposing pump electrode and the reference electrode by the concentration calculating part.

The gas sensor 100 is installed in a piping or the like so that the front end part of the sensor element 101 comes into contact with a measurement-object gas that contains water vapor. The measurement-object gas normally contains oxygen gas as well. The gas sensor 100 is started when, for example, the gas sensor 100 receives a start signal (Dew point). When the gas sensor 100 is installed in a car or the like, the start signal (Dew point) is, for example, a signal sent from an ECU, an exhaust gas treatment system, or the like of the car to the gas sensor 100. The gas sensor 100 may be started by, for example, manually turning on the power supply of the control unit 90.

When the gas sensor 100 is started, the heater control part 92 of the control part 91 heats the heater 72 and maintains the temperature of the heater 72 at a desired temperature. Thus, the sensor element 101 is maintained at a driving temperature (e.g., about 800° C.) at which the concentration of H₂O is measured with high accuracy due to the activation of the solid electrolyte.

Next, the pump control part 93 performs the control of the main pump cell 21 and the decomposing pump cell 50 as described above. The pump control part 93 may start the control after the sensor element 101 reaches the driving temperature, or may start the control at a temperature lower than the driving temperature.

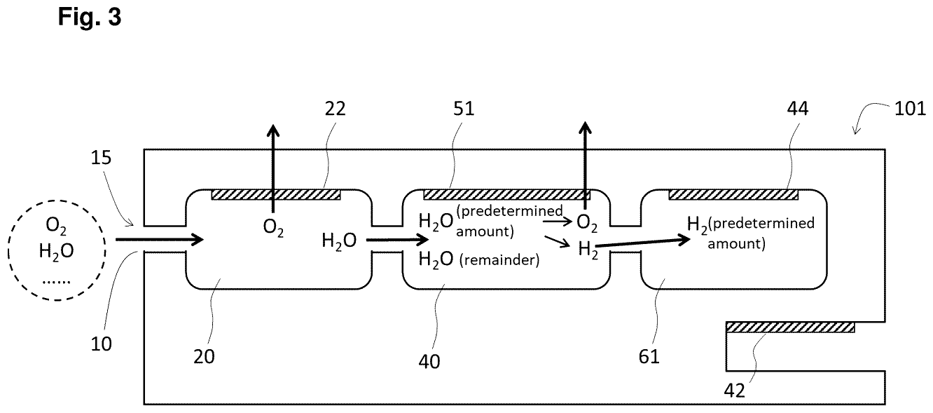

FIG. 3 is a conceptual diagram showing a principle of measuring water vapor in the gas sensor 100. Focusing on the behavior of water vapor H₂O and oxygen O₂ in the measurement-object gas, only water vapor H₂O and oxygen O₂ are shown in FIG. 3. Also, since structure of the sensor element 101 is illustrated in a simplified manner in FIG. 3, description will be made while also referring to FIG. 1 accordingly. The measurement-object gas is introduced from the gas inlet 10, passes through the first diffusion-rate limiting part 11, the buffer space 12, and the second diffusion-rate limiting part 13 in this order, and reaches the first internal cavity 20. The oxygen concentration is adjusted to a predetermined concentration by the action of the main pump cell 21. Consequently, the oxygen concentration in the first internal cavity 20, that is, the oxygen concentration in the measurement-object gas to be introduced in the second internal cavity 40 may be adjusted to a low concentration that does not substantially affect measurement of water vapor H₂O. For example, the oxygen concentration may be adjusted so that almost all of oxygen gas in the measurement-object gas is pumped out and water vapor in the measurement-object gas is not substantially decomposed. Then, the measurement-object gas whose oxygen concentration has been adjusted to the predetermined concentration passes through the third diffusion-rate limiting part 30 and reaches the second internal cavity 40.

In the second internal cavity 40, at least a part of water vapor H₂O contained in the reaching measurement-object gas is decomposed (or, reduced) at the decomposing pump electrode 51 to generate hydrogen and oxygen (2H₂O→2H₂+O₂). The oxygen generated by the decomposition of water vapor is pumped out from the second internal cavity 40 by the decomposing pump cell 50. Then, the measurement-object gas containing hydrogen gas H₂ generated by the decomposition of water vapor passes through the fourth diffusion-rate limiting part 60 to be introduced into the third internal cavity 61, and reaches the detection electrode 44. It is considered that water vapor H₂O that is not decomposed at the decomposing pump electrode 51 is also contained in the measurement-object gas reaching the detection electrode 44.

As described above, the pump control part 93 performs control so that the voltage V2 between the detection electrode 44 and the reference electrode 42 in the electromotive force detection sensor cell 82 is at the predetermined value (namely, the set value V2_{SET}). The voltage V2 is the electromotive force generated in accordance with concentrations of oxygen gas and hydrogen gas in the atmosphere around the detection electrode 44, as described above. Water vapor H₂O itself is considered not to affect the value of the voltage V2. The oxygen concentration in the measurement-object gas is adjusted to the predetermined concentration by the action of the main pump cell 21, as described above. Therefore, it is considered that performing the control so the voltage V2 is at the predetermined value can be rephrased as performing the control so that the amount (or the number) of hydrogen gas in the measurement-object gas reaching the detection electrode 44 is a predetermined amount (or a predetermined number).

That is, the pump control part 93 operates the decomposing pump cell 50 so that an amount of hydrogen gas in the measurement-object gas reaching the detection electrode 44 is a predetermined amount, and makes control so that water vapor is decomposed at the decomposing pump electrode 51 to generate the predetermined amount of hydrogen gas. In other words, the pump control part 93 makes the control to decompose the predetermined amount of water vapor, regardless of water vapor concentration in the measurement-object gas. For example, when water vapor concentration in the measurement-object gas is high, water vapor may be decomposed at a low decomposition rate at the decomposing pump electrode 51. On the other hand, when water vapor concentration in the measurement-object gas is low, water vapor may be decomposed at a high decomposition rate at the decomposing pump electrode 51.

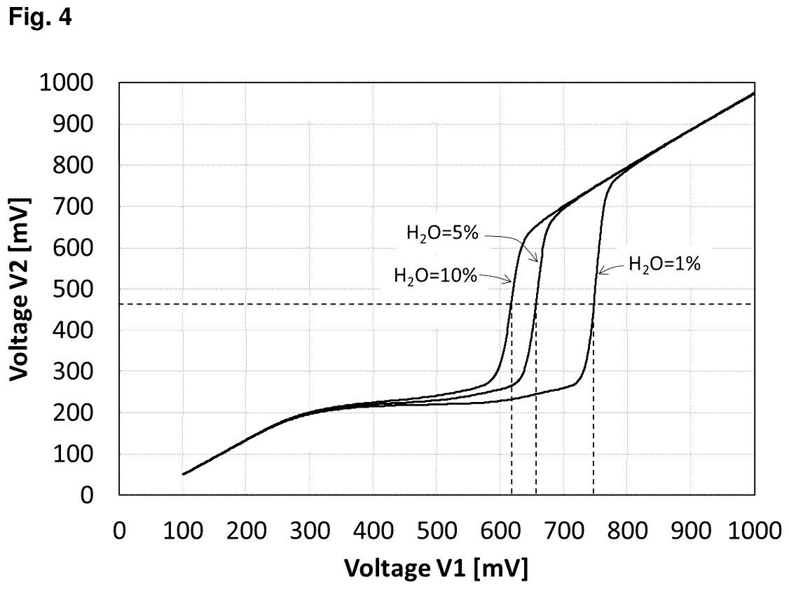

FIG. 4 is a graph showing one example of a relationship between the voltage V1 in the oxygen-partial-pressure detection sensor cell 81 for decomposing pump control and the voltage V2 in the electromotive force detection sensor cell 82. The horizontal axis of the graph represents the voltage V1 (mV) and the vertical axis of the graph represents the voltage V2 (mV). FIG. 4 shows relationships between the voltage V1 and the voltage V2 when water vapor concentrations are H₂O=1%, 5%, and 10% (volume basis, the same hereafter), respectively. It was found that in each of the concentrations, a region where change in the voltage V1 is small with respect to change in the voltage V2 is present. In this region, it was found that the lower the water vapor concentration, the larger the voltage V1 tends to be when comparing the voltage V1 at the same voltage V2 (e.g., at the voltage V2=450 mV).

When the value of V2 is the same, as described above, the amount of water vapor decomposed at the decomposing pump electrode 51 is considered to be the same, regardless of water vapor concentration in the measurement-object gas. Therefore, the lower the voltage V1 between the decomposing pump electrode 51 and the reference electrode 42 in the oxygen-partial-pressure detection sensor cell 81 for decomposing pump control, the lower the decomposition rate at the decomposing pump electrode 51 is considered to be. Further, the higher the voltage V1, the higher the decomposition rate at the decomposing pump electrode 51 is considered to be.

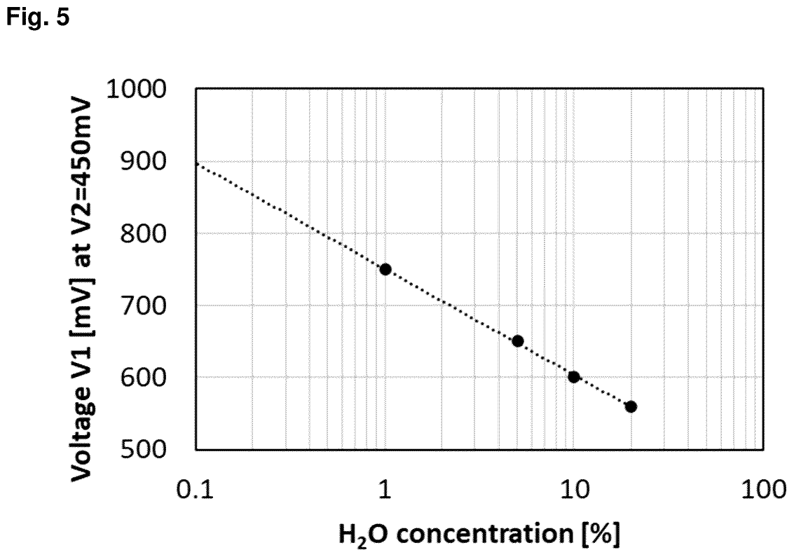

FIG. 5 is a graph showing one example of a relationship between H₂O concentration in the measurement-object gas and the voltage V1 in the gas sensor 100. The horizontal axis of the graph represents the H₂O concentration (%) and the vertical axis of the graph represents the voltage V1 (mV) when the voltage V2 is to be at 450 mV. Thus, it was found that there is a correlation between H₂O concentration and the voltage V1. By using such a correlation, the concentration calculating part 94 can calculate a water vapor concentration in the measurement-object gas based on the voltage V1.

Further, as shown in FIG. 5, the lower the H₂O concentration in the measurement-object gas is, the larger the change of the voltage V1 with respect to the H₂O concentration change is. That is, the lower the H₂O concentration in the measurement-object gas is, the more greatly the voltage V1 changes with respect to a minute change in the H₂O concentration, and therefore resolution of the measurement is tend to be higher. Thus, in case of measuring the measurement-object gas that contains low concentration of H2O, H₂O concentration can be measured with particularly high measurement accuracy.

### Variation

Hereinafter, an example of another embodiment of a gas sensor of the present invention will be described. In a gas sensor of a variation,
the sensor element further includes a current measurement pump cell including the intracavity detection electrode, and an extracavity detection electrode disposed at a position different from the measurement-object gas flow cavity on the base part and corresponding to the intracavity detection electrode; and
the control unit further includes a switching unit for switching whether a current flows through the current measurement pump cell or not.

In the above gas sensor 100, H₂O concentration in the measurement-object gas is calculated based on the voltage V1 between the decomposing pump electrode 51 and the reference electrode 42. In a gas sensor 200 of a variation, measurement is made in a voltage measurement mode in which H₂O concentration in the measurement-object gas is calculated based on the voltage V1 as in the case of the gas sensor 100, or in a current measurement mode in which H₂O concentration in the measurement-object gas is calculated based on a pump current flowing through the current measurement pump cell, while switching between the voltage measurement mode and the current measurement mode.

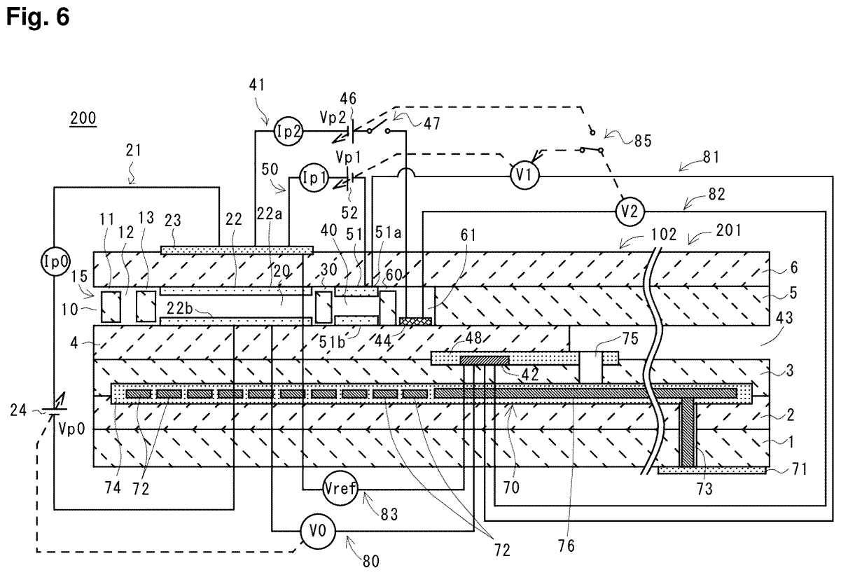

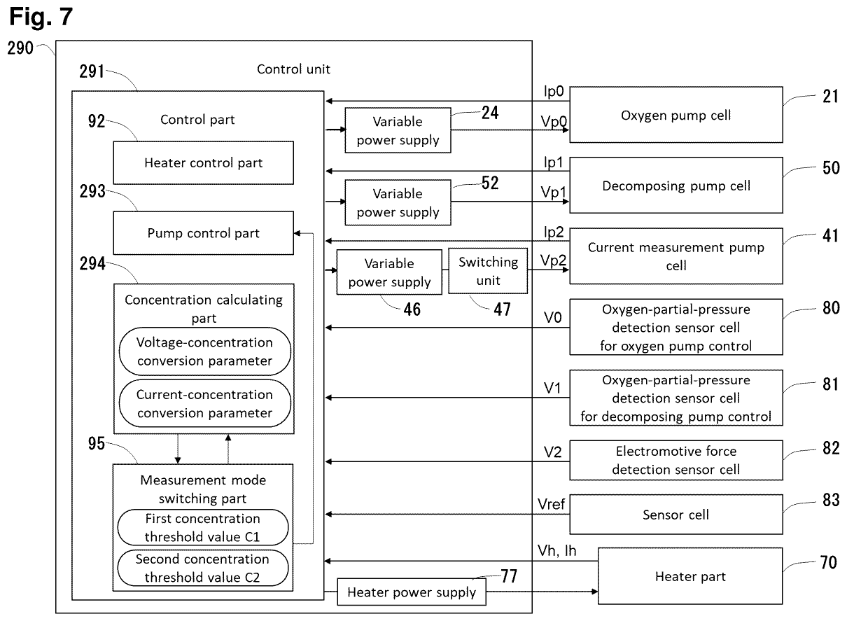

FIG. 6 is a vertical sectional schematic view in the longitudinal direction, showing one example of a general configuration of the gas sensor 200 including a sensor element 201. In FIG. 6, the same member as in FIG. 1 is denoted by the same sign, and description of the same member is omitted. FIG. 7 is a block diagram showing electric connections between the control unit 290 and the sensor element 201. In FIG. 7, the same member as in FIG. 2 is denoted by the same sign, and description of the same member is omitted. The control unit 290 includes a switching unit 47 for switching whether a current flows through a current measurement pump cell 41 or not.

In the gas sensor 200, the switching unit 47 may have a mechanism for switching whether a current flows through the current measurement pump cell 41 or not. For example, the switching unit 47 may be a switch 47 as illustrated in FIG. 6, or a mechanism including a switch. The switch may be a contact switch that mechanically opens and closes a contact on an electrical circuit, or may be a switch using a switching element that turn on and off the current flowing in the electrical circuit. The switching element includes a diode, a thyristor, a transistor, a MOSFET, and the like. The configuration of the switch may appropriately be determined by a person skilled in the art. By turning the switch OFF, it is possible to cut off a conduction in the current measurement pump cell 41 to switch so that a current does not flow through the current measurement pump cell 41. By turning the switch ON, it is possible to conduct the current measurement pump cell 41 to switch so that a current flows through the current measurement pump cell 41.

Alternatively, for example, the switching unit 47 may be a variable power supply 46, or a mechanism including the variable power supply 46. By setting a voltage in the variable power supply 46 to zero, it is possible to not to apply a voltage in the current measurement pump cell to switch so that a current does not flow through the current measurement pump cell. By setting a voltage in the variable power supply 46 to a predetermined value, it is possible to apply the voltage in the current measurement pump cell to switch so that a current flows through the current measurement pump cell.

The sensor element 201 includes the current measurement pump cell 41. The current measurement pump cell 41 is an electrochemical pump cell including the intracavity detection electrode (in this embodiment, the detection electrode 44) disposed at a position farther from the front end portion in the longitudinal direction of the base part 102 than the intracavity decomposing electrode (in this embodiment, the decomposing pump electrode 51) in the measurement-object gas flow cavity 15, and an extracavity detection electrode disposed at a position different from the measurement-object gas flow cavity 15 on the base part 102 and corresponding to the intracavity detection electrode. In the gas sensor 200, the outer pump electrode 23 disposed on the outer surface of the base part 102 functions also as the extracavity detection electrode. The phrase “corresponding to the intracavity detection electrode” means that the outer pump electrode 23 and the detection electrode 44 are provided with the second solid electrolyte layer 6, the spacer layer 5, and the first solid electrolyte layer 4 being interposed therebetween.

That is, the current measurement pump cell 41 is an electrochemical pump cell composed of the detection electrode 44 disposed on the upper surface of the first solid electrolyte layer 4 facing with the third internal cavity 61, the outer pump electrode 23 (the outer electrode is not limited to the outer pump electrode 23, but may be any suitable electrode disposed at a position different from the measurement-object gas flow cavity 15), the second solid electrolyte layer 6, the spacer layer 5, and the first solid electrolyte layer 4.

In the current measurement pump cell 41, by applying a voltage Vp2 between the detection electrode 44 and the outer pump electrode 23 by a variable power supply 46 to make a pump current Ip2 flow between the detection electrode 44 and the outer pump electrode 23, it is possible to pump oxygen into the third internal cavity 61.

The current measurement pump cell 41 is configured to switch whether a current flows through the current measurement pump cell 41 or not by the switching unit 47. Hereinafter, the gas sensor 200 in which the switching unit 47 is a switch is described as an example with reference to FIG. 6. The switching unit 47 is schematically illustrated in FIG. 6 using a circuit symbol of a contact switch, but the switching unit 47 may be a switch using a switching element. A location of the switching unit 47 is not limited to the position illustrated in FIG. 6, but the switching unit 47 can be located anywhere on the circuit of the current measurement pump cell 41.

When the switching unit 47 is turned OFF, electrical connection of the current measurement pump cell 41 is cut off so that a current does not flow through the current measurement pump cell 41. The phrase “a current does not flow” means that a current value in the current measurement pump cell 41 is zero or substantially zero.

In this case, the above-described feedback control is performed for adjusting a value of the voltage V1 in the oxygen-partial-pressure detection sensor cell 81 for decomposing pump control based on the voltage V2 detected in the electromotive force detection sensor cell 82. And, the voltage measurement mode in which H₂O concentration in the measurement-object gas is calculated based on the voltage V1 is executed.

When the switching unit 47 is turned ON, the current measurement pump cell 41 is electrically connected so that a current flows through the current measurement pump cell 41.

In this case, the pump voltage Vp2 of the variable power supply 46 is controlled based on the voltage V2 detected in the electromotive force detection sensor cell 82. As a result, the pump current Ip2 flows through the current measurement pump cell 41. And, the current measurement mode in which H₂O concentration in the measurement-object gas is calculated based on the pump current Ip2 is executed.

Accordingly, in the gas sensor 200, objects to which the value of the voltage V2 is fed back are different between when the switching unit 47 is turned OFF (namely, the voltage measurement mode) and when the switching unit 47 is turned ON (namely, the current measurement mode). That is, the value of the voltage V2 is fed back to the voltage V1 in the voltage measurement mode, and the value of the voltage V2 is fed back to the pump voltage Vp2 in the current measurement mode. A feed back switching part 85 in FIG. 6 conceptually indicates that the object to which the value of the voltage V2 is fed back is switched according to the measurement mode, and does not exist as an entity. The object to which the value of the voltage V2 is fed back is set in a control program for each of the measurement modes by a pump control part 293 which is described below.

FIG. 7 is a block diagram showing electric connections between the control unit 290 and the respective pump cells 21, 50, and 41, the respective sensor cells 80, 81, 82, and 83, and the heater part 70 of the sensor element 201, in the gas sensor 200 of the variation. In the gas sensor 200 of the variation, the control unit 290 includes the variable power supplies 24, 46, and 52, the switching unit 47 for switching whether a current flows through the current measurement pump cell 41 or not, and a control part 291. The control part 291 includes the heater control part 92, the pump control part 293, a concentration calculating part 294, and a measurement mode switching part 95. The switching unit 47 is a component that receives a control signal from the measurement mode switching part 95 and switches whether or not to apply current to the current measurement pump cell 41. In FIG. 7, the same member as in FIG. 2 is denoted by the same sign, and description of the same member is omitted.

The pump control part 293 has:
a pump control in a voltage measurement mode in which H₂O concentration in a measurement-object gas is calculated based on the voltage V1 between the decomposing pump electrode 51 and the reference electrode 42; and
a pump control in a current measurement mode in which H₂O concentration in the measurement-object gas is calculated based on the pump current Ip2 flowing through the current measurement pump cell 41.

Regardless of the measurement modes, the pump control part 293 adjusts an oxygen concentration in a measurement-object gas to a predetermined concentration by the oxygen pump cell (namely, the main pump cell 21) as with the case of the above-described gas sensor 100.

In the voltage measurement mode, the pump control part 293 performs, as with the case of the pump control part 93 in the above-described gas sensor 100, feedback control for adjusting the target value (namely, the set value V1_{SET}) of the voltage V1 in the oxygen-partial-pressure detection sensor cell 81 for decomposing pump control so that the voltage V2 between the detection electrode 44 and the reference electrode 42 in the electromotive force detection sensor cell 82 is at the target value (namely, the set value V2_{SET}). The pump control part 293 also performs feedback control of the pump voltage Vp1 of the variable power supply 52 in the decomposing pump cell 50 so that the voltage V1 in the oxygen-partial-pressure detection sensor cell 81 for decomposing pump control is at the adjusted target value (namely, the set value V1_{SET}).

In the current measurement mode, the pump control part 293 performs feedback control of the pump voltage Vp1 of the variable power supply 52 in the decomposing pump cell 50 so that the voltage V1 in the oxygen-partial-pressure detection sensor cell 81 for decomposing pump control is at a target value (referred to as a set value V1a_{SET}). The set value V1a_{SET} may be set as a value such that substantially all of water vapor in the measurement-object gas is decomposed (reduced) at the decomposing pump electrode 51. The set value V1a_{SET} is normally set as a larger value than the above-described set value V0_{SET} in the control of the main pump cell 21. Oxygen generated by the decomposition of water vapor is pumped out by making the pump current Ip1 flow through the decomposing pump cell 50. Then, the measurement-object gas which contains hydrogen generated by the decomposition of water vapor is introduced into the third internal cavity 61.

In the current measurement mode, the pump control part 293 also performs feedback control of the pump voltage Vp2 of the variable power supply 46 in the current measurement pump cell 41 so that the voltage V2 between the detection electrode 44 and the reference electrode 42 in the electromotive force detection sensor cell 82 is at a target value (referred to as a set value V2a_{SET}). The set value V2a_{SET} may be set as a value such that substantially all of hydrogen generated by the decomposition of water vapor at the decomposing pump electrode 51 is burned (oxidized) at the detection electrode 44. The pump control part 293 pumps in oxygen in an amount for burning (oxidizing) substantially all of the hydrogen generated by the decomposition of water vapor at the detection electrode 44 by making the pump current Ip2 flow through the current measurement pump cell 41. Therefore, the pump current Ip2 becomes at a value corresponding to an amount (or, a concentration) of hydrogen generated by the decomposition of water vapor.

When substantially all of water vapor in the measurement-object gas is decomposed (reduced) at the decomposing pump electrode 51 (2H₂O→2H₂+O₂), hydrogen gas is generated in an amount proportional to an amount (or, a concentration) of the water vapor in the measurement-object gas. An amount of oxygen pumped in as the pump current Ip2 in order to burn substantially all of the hydrogen gas at the detection electrode 44 is proportional to the amount of the hydrogen gas. Therefore, a linear relationship exists between H₂O concentration in the measurement-object gas and the pump current Ip2, where magnitude (an absolute value) of the pump current Ip2 increases in accordance with the H₂O concentration. Based on this linear relationship, measurement can be performed with high resolution regardless of the H₂O concentration.

In the current measurement mode, even when H₂O concentration in the measurement-object gas is at zero, the pump current Ip2 is not at zero and a minute current flows. This minute current is referred to as an offset current Ip2offset. The offset current Ip2offset is a minute current that flows with being affected by the control in the main pump cell 21 and the decomposing pump cell 50, regardless of H₂O concentration. If the current value of the offset current Ip2offset changes for some reasons, the pump current Ip2 detected in the current measurement pump cell 41 shifts by a change amount ΔIp2offset of the offset current Ip2offset, regardless of the H₂O concentration in the measurement-object gas. The offset current Ip2offset is considered to change due to, for example, change in the electrode temperature by change in the measurement-object gas temperature, and change in the output of heater power supply 77 therewith.

In case of measuring the measurement-object gas that contains high concentration of H₂O, magnitude (an absolute value) of the pump current Ip2 detected in the current measurement pump cell 41 is relatively large so that the change in the pump current Ip2 due to the change ΔIp2offset of the offset current Ip2offset is relatively small. Therefore, even if the offset current Ip2offset changes, H₂O concentration can be measured with higher measurement accuracy. That is, in case of measuring the measurement-object gas that contains high concentration of H₂O, H₂O concentration can be measured with higher measurement accuracy by using the current measurement mode.

On the other hand, as described above, regarding the voltage V1 between the decomposing pump electrode 51 and the reference electrode 42 in the voltage measurement mode, as shown in FIG. 5, the lower the H₂O concentration in the measurement-object gas is, the larger the change of the voltage V1 with respect to the H₂O concentration change is. That is, the lower the H₂O concentration in the measurement-object gas is, the more greatly the voltage V1 changes with respect to a minute change in the H₂O concentration, and therefore resolution of the measurement is tend to be higher. Thus, in case of measuring the measurement-object gas that contains low concentration of H₂O, H₂O concentration can be measured with higher measurement accuracy by using the voltage measurement mode.

The concentration calculating part 294 is configured to calculate a concentration of a target gas to be measured (in the gas sensor 100 of the variation, a H₂O concentration) in a measurement-object gas.

In the voltage measurement mode, as in the case of the concentration calculating part 94 in the above-described gas sensor 100, the concentration calculating part 294 acquires the voltage V1 between the intracavity decomposing pump electrode (the decomposing pump electrode 51) and the reference electrode 42 in the oxygen-partial-pressure detection sensor cell 81 for decomposing pump control, and calculates the H₂O concentration in the measurement-object gas on the basis of the previously-stored conversion parameter (voltage-concentration conversion parameter) between the voltage V1 and the H₂O concentration in the measurement-object gas. And, the concentration calculating part 294 outputs the calculated H₂O concentration as a measurement value of the gas sensor 200.

In the current measurement mode, the concentration calculating part 294 acquires the pump current Ip2 in the current measurement pump cell 41, and calculates the H₂O concentration in a measurement-object gas on the basis of a previously-stored conversion parameter (current-concentration conversion parameter) between the pump current Ip2 and the H₂O concentration in the measurement-object gas. And, the concentration calculating part 294 outputs the H₂O concentration as a measurement value of the gas sensor 200. The current-concentration conversion parameter is previously stored in the memory of the control part 291 which functions as the concentration calculating part 294. The current-concentration conversion parameter may appropriately be determined by those skilled in the art by, for example, previously performing an experiment on the gas sensor 200. The current-concentration conversion parameter may be, for example, the coefficient of an approximate expression (e.g., linear function) obtained by experiment or a map showing the relationship between the pump current Ip2 and the H₂O concentration in a measurement-object gas. The current-concentration conversion parameter may be specific to each individual gas sensor 200 or may be common to a plurality of gas sensors.

The measurement mode switching part 95 is configured to perform switching between the voltage measurement mode and the current measurement mode that are described above.

The measurement mode switching part 95 switches the switching unit 47 so that a current does not flow or does not substantially flow through the current measurement pump cell 41 in case of switching from the current measurement mode to the voltage measurement mode. In the gas sensor 200 of the variation, the measurement mode switching part 95 outputs a control signal to the switching unit 47 (a switching switch) to be OFF. Further, the measurement mode switching part 95 gives instructions to the pump control part 293 to perform the control in the voltage measurement mode. In this case, the pump control part 293 performs the above-described feedback control for adjusting a value of the voltage V1 between the decomposing pump electrode 51 and the reference electrode 42 in the oxygen-partial-pressure detection sensor cell 81 for decomposing pump control on the basis of the voltage V2 detected in the electromotive force detection sensor cell 82. The measurement mode switching part 95 gives instructions to the concentration calculating part 294 to acquire the voltage V1 between the decomposing pump electrode 51 and the reference electrode 42, and calculate the H₂O concentration on the basis of the voltage-concentration conversion parameter.

The measurement mode switching part 95 switches the switching unit 47 so that a current flows through the current measurement pump cell 41 in case of switching from the voltage measurement mode to the current measurement mode. In the gas sensor 200 of the variation, the measurement mode switching part 95 outputs a control signal to the switching unit 47 (the switching switch) to be ON. Further, the measurement mode switching part 95 gives instructions to the pump control part 293 to perform the control in the current measurement mode. In this case, the pump control part 293 performs the above-described feedback control to the pump voltage Vp2 of the variable power supply 46 in the current measurement pump cell 41, and detects the pump current Ip2 in the current measurement pump cell 41. The measurement mode switching part 95 gives instructions to the concentration calculating part 294 to acquire the pump current Ip2 in the current measurement pump cell 41, and calculate the H₂O concentration on the basis of the current-concentration conversion parameter.

Switching between the voltage measurement mode and the current measurement mode may be performed on the basis of a concentration of a target gas to be measured (in the gas sensor 200, a H₂O concentration) output by the concentration calculating part 294. The measurement mode switching part 95 (more specifically, the memory that functions as the measurement mode switching part 95 in the control unit 291) may store in advance, a first concentration threshold value C1 that is a threshold value for switching from the current measurement mode to the voltage measurement mode, and a second concentration threshold value C2 that is a threshold value for switching from the voltage measurement mode to the current measurement mode. The measurement mode switching part 95 may continuously acquire the H₂O concentration output by the concentration calculating part 294, or may acquire the H₂O concentration output by the concentration calculating part 294 at predetermined intervals.

The measurement mode switching part 95 may acquire the H₂O concentration output by the concentration calculating part 294 in the current measurement mode, and may switch to the voltage measurement mode when the measurement mode switching part 95 determines that H₂O concentration is in a low concentration range where H₂O concentration is lower than a predetermined first concentration threshold value C1. The measurement mode switching part 95 may maintain the current measurement mode when the measurement mode switching part 95 determines that the H₂O concentration is equal to or higher than the predetermined first concentration threshold value C1.

The measurement mode switching part 95 may also acquire the H₂O concentration output by the concentration calculating part 294 in the voltage measurement mode, and may switch to the current measurement mode when the measurement mode switching part 95 determines that H₂O concentration is in a high concentration range where H₂O concentration is higher than a predetermined second concentration threshold value C2. The measurement mode switching part 95 may maintain the voltage measurement mode when the measurement mode switching part 95 determines that the H₂O concentration is equal to or lower than the predetermined second concentration threshold value C2.

The first concentration threshold value C1 that is the threshold value for switching from the current measurement mode to the voltage measurement mode may appropriately be determined by a person skilled in the art. The first concentration threshold value C1 may be a different value depending on the assumed concentration range of H₂O in the measurement-object gas and the measurement accuracy required for the gas sensor 200. The first concentration threshold value C1 may be a lower limit of the H₂O concentration at which a desired measurement accuracy can be obtained when measuring in the current measurement mode. The first concentration threshold value C1 may be, for example, a lower limit of the H₂O concentration at which a value of the offset current Ip2offset is in an acceptable range in terms of measurement accuracy. Alternatively, the first concentration threshold value C1 may be, for example, a lower limit of the H₂O concentration at which a value of the change ΔIp2offset of the offset current Ip2offset is in an acceptable range in terms of measurement accuracy. The first concentration threshold value C1 may be, for example, in a rage of 0.1% to 10%. For example, the first concentration threshold value C1 may be 3%.

The second concentration threshold value C2 that is the threshold value for switching from the voltage measurement mode to the current measurement mode may appropriately be determined by a person skilled in the art. The second concentration threshold value C2 may be a different value depending on the assumed concentration range of H₂O in the measurement-object gas and the measurement accuracy required for the gas sensor 200. The second concentration threshold value C2 may be an upper limit of the H₂O concentration at which a desired measurement accuracy can be obtained when measuring in the voltage measurement mode. The second concentration threshold value C2 may be, for example, an upper limit of the H₂O concentration at which a change amount of the voltage V1 with respect to a change amount of the H₂O concentration is in an acceptable range in terms of the resolution of the measurement. The second concentration threshold value C2 may be, for example, in a rage of 0.1% to 10%. For example, the second concentration threshold value C2 may be 5%.

The first concentration threshold value C1 and the second concentration threshold value C2 may be the same value, or may be different from each other. When the first concentration threshold value C1 and the second concentration threshold value C2 are the same value, the voltage measurement mode may be executed in the low concentration range where H₂O concentration is lower than the first concentration threshold value C1(=the second concentration threshold value C2), and the current measurement mode may be executed in the high concentration range where H₂O concentration is equal to or higher than the first concentration threshold value C1(=the second concentration threshold value C2).

It is more preferred that the first concentration threshold value C1 is lower than the second concentration threshold value C2. In other words, it is more preferred that two threshold values that have a concentration range are used.

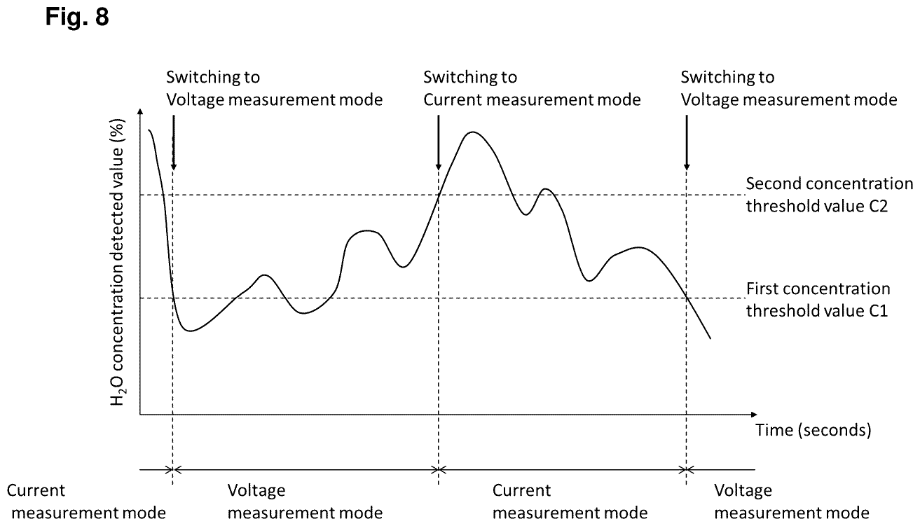

FIG. 8 is a graph schematically showing one example of time variation of H₂O concentration detected value output by the gas sensor 200 and switching of the measurement modes. The horizontal axis of the graph represents time (seconds) and the vertical axis of the graph represents the H₂O concentration detected value (%) output by the gas sensor 200.

As shown in FIG. 8, when H₂O concentration in the measurement-object gas (i.e., H₂O concentration output by the gas sensor 200) falls below the first concentration threshold value C1, the measurement mode switching part 95 switches to the voltage measurement mode. Even if the H₂O concentration in the measurement-object gas fluctuates around the first concentration threshold value C1 and exceeds the first concentration threshold value C1 after switching to the voltage measurement mode, the voltage measurement mode is continued without switching to the current measurement mode until the H₂O concentration in the measurement-object gas exceeds the second concentration threshold value C2. After that, when the H₂O concentration in the measurement-object gas exceeds the second concentration threshold value C2, the measurement mode switching part 95 switches to the current measurement mode. Even if the H₂O concentration in the measurement-object gas fluctuates around the second concentration threshold value C2 and falls below the second concentration threshold value C2 after switching to the current measurement mode, the current measurement mode is continued without switching to the voltage measurement mode until the H₂O concentration in the measurement-object gas falls below the first concentration threshold value C1.

Thus, when the first concentration threshold value C1 is lower than the second concentration threshold value C2, the concentration range (intermediate concentration range) between the first concentration threshold value C1 and the second concentration threshold value C2 serves as a buffer range where the most recent measurement mode continues to be maintained without switching measurement modes. That is, when the H₂O concentration in the measurement-object gas frequently fluctuates around the first concentration threshold value C1 or the second concentration threshold value C2, switching of the measurement modes can be adjusted not to be too frequent. At the time of switching the measurement mode, the control of the current measurement pump cell 41 is changed, and thereby a situation where the gas sensor 200 cannot measure H₂O concentration temporarily may occur. By providing the buffer range between the first concentration threshold value C1 and the second concentration threshold value C2, switching of the measurement modes can be adjusted not to occur too frequent. Accordingly, the gas sensor 200 can measure H₂O concentration more continuously and accurately.

For example, the first concentration threshold value C1 may be 0.1% to 5%, and the second concentration threshold value C2 may be 5% to 10%. For example, the first concentration threshold value C1 may be 3%, and the second concentration threshold value C2 may be 5%.

The measurement mode may be switched in the buffer range between the first concentration threshold value C1 and the second concentration threshold value C2. The measurement mode may be switched, for example, by predicting H₂O concentration detected value based on time variation of the H₂O concentration detected value (slope of the graph in FIG. 8). The measurement mode may be switched, for example, based on the time for which the H₂O concentration detected value is within the buffer range.

### (Detection of Concentration of Target Gas to be Measure in Variation)

Next, a method for measuring a concentration of the target gas to be measured in the measurement-object gas by using the gas sensor 200 will be described.

A control method of the gas sensor of the variation includes:
a step of performing a voltage measurement mode in which the pump control step and the concentration calculating step are performed to calculate a concentration of the target gas to be measured in the measurement-object gas based on the value of the voltage between the intracavity decomposing pump electrode and the reference electrode, or a current measurement mode in which the oxygen pump cell, the decomposing pump cell and the current measurement pump cell are operated to calculate a concentration of the target gas to be measured in the measurement-object gas based on a current value in the current measurement pump cell, while switching between the voltage measurement mode and the current measurement mode by using the switching unit. In the above step, any one of the measurement modes is always executed to continuously detect the concentration.

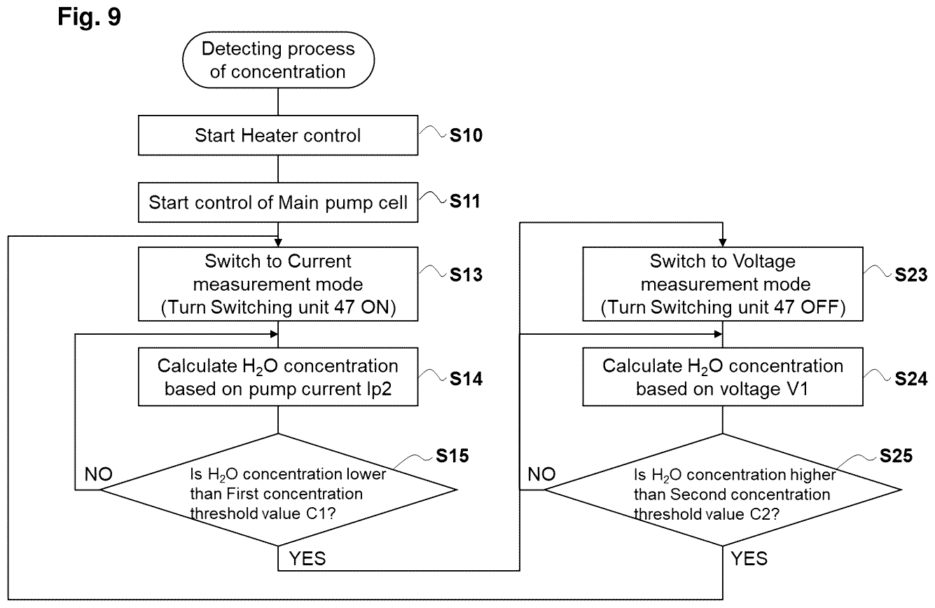

The detecting process of H₂O concentration in the gas sensor 200 of the variation will be described below in detail. FIG. 9 is a flow chart showing one example of detecting process of H₂O concentration in the gas sensor 200 of the variation.

The detecting process of H₂O concentration is started when, for example, the gas sensor 200 receives a start signal (Dew point). When the gas sensor 200 is installed in a car or the like, the start signal (Dew point) is, for example, a signal sent from an ECU, an exhaust gas treatment system, or the like of the car to the gas sensor 200. The detecting process of H₂O concentration may be started by, for example, manually turning on the power supply of the control unit 290.

When the detecting process of H₂O concentration is started, the heater control part 92 of the control part 291 starts to heat the heater 72 by the application of power to the heater 72 (step S10), and the sensor element 201 is maintained at a driving temperature (e.g., about 800° C.) at which the concentration of H₂O is measured with high accuracy due to the activation of the solid electrolyte.

Next, the pump control part 293 starts to control the main pump cell 21 (step S11). Specifically, the feedback control based on the set value V0_{SET} is performed for the main pump cell 21. The step S11 may be performed after the sensor element 101 reaches the driving temperature, or may be performed at a temperature lower than the driving temperature.

Next, the measurement mode switching part 95 of the control part 291 performs switching to the current measurement mode (step S13). Specifically, the measurement mode switching part 95 outputs a control signal to the switching unit 47 to be ON. Further, the measurement mode switching part 95 gives instructions to the pump control part 293 to perform the control in the current measurement mode. In this case, the pump control part 293 performs the feedback control to the decomposing pump cell 50 on the basis of the set value V1a_{SET}, and the feedback control to the pump voltage Vp2 of the variable power supply 46 in the current measurement pump cell 41 on the basis of the set value V2a_{SET}, and detects the pump current Ip2 in the current measurement pump cell 41. The measurement mode switching part 95 gives instructions to the concentration calculating part 294 to acquire the pump current Ip2 in the current measurement pump cell 41, and calculate the H₂O concentration on the basis of the current-concentration conversion parameter. In the current measurement mode, as described above, the pump current Ip2 corresponding to the H₂O concentration flows through the current measurement pump cell 41. The step S13 may be performed simultaneously with the above-described step S11.

Then, the concentration calculating part 294 acquires the pump current Ip2 in the current measurement pump cell 41, and calculates the H₂O concentration in the measurement-object gas on the basis of the previously-stored conversion parameter (current-concentration conversion parameter) between the pump current Ip2 and the H₂O concentration in the measurement-object gas (step S14). The calculated H₂O concentration is output as the detected value of the gas sensor 200. After the step S14, the measurement mode switching part 95 acquires the H₂O concentration calculated by the concentration calculating part 294, and determines whether or not the acquired H₂O concentration is lower than the first concentration threshold value C1 (step S15). As the first concentration threshold value C1, for example, the lower limit of the H₂O concentration at which the desired measurement accuracy can be obtained when measuring in the current measurement mode is set in advance.

In the step S15, when the H₂O concentration acquired from the concentration calculating part 294 is equal to or higher than the first concentration threshold value C1, the step S14 and subsequent processes are performed. That is, when the H₂O concentration is equal to or higher than the first concentration threshold value C1, the measurement mode switching part 95 does not switch the measurement mode, and the pump control part 293 and the concentration calculating part 294 continue the current measurement mode.

In the step S15, when the H₂O concentration acquired from the concentration calculating part 294 is lower than the first concentration threshold value C1, the measurement mode switching part 95 performs switching to the voltage measurement mode (step S23). Specifically, the measurement mode switching part 95 outputs a control signal to the switching unit 47 to be OFF. Further, the measurement mode switching part 95 gives instructions to the pump control part 293 to perform the control in the voltage measurement mode. In this case, the pump control part 293 performs the feedback control for adjusting the set value V1_{SET} of the voltage V1 in the oxygen-partial-pressure detection sensor cell 81 for decomposing pump control on the basis of the set value V2_{SET} of the voltage V2 in the electromotive force detection sensor cell 82. The measurement mode switching part 95 gives instructions to the concentration calculating part 294 to acquire the voltage V1 in the oxygen-partial-pressure detection sensor cell 81 for decomposing pump control, and calculate the H₂O concentration on the basis of the voltage-concentration conversion parameter. In the voltage measurement mode, the pump current Ip2 does not flow through the current measurement pump cell 41.

Then, the concentration calculating part 294 acquires the voltage V1 in the oxygen-partial-pressure detection sensor cell 81 for decomposing pump control, and calculates the H₂O concentration in the measurement-object gas on the basis of the previously-stored conversion parameter (voltage-concentration conversion parameter) between the voltage V1 and the H₂O concentration in the measurement-object gas (step S24). The calculated H₂O concentration is output as the detected value of the gas sensor 200. After the step S24, the measurement mode switching part 95 acquires the H₂O concentration calculated by the concentration calculating part 294, and determines whether or not the acquired H₂O concentration is higher than the second concentration threshold value C2 (step S25). As the second concentration threshold value C2, for example, the upper limit of the H₂O concentration at which the desired measurement accuracy can be obtained when measuring in the voltage measurement mode is set in advance.

In the step S25, when the H₂O concentration acquired from the concentration calculating part 294 is equal to or lower than the second concentration threshold value C2, the step S24 and subsequent processes are performed. That is, when the H₂O concentration is equal to or lower than the second concentration threshold value C2, the measurement mode switching part 95 does not switch the measurement mode, and the pump control part 293 and the concentration calculating part 294 continue the voltage measurement mode.

In the step S25, when the H₂O concentration acquired from the concentration calculating part 294 is higher than the second concentration threshold value C2, the measurement mode switching part 95 performs switching to the current measurement mode (step S13), and the step S14 and subsequent processes are performed.

Accordingly, the control part 291 determines, in the measurement mode switching part 95, whether to use the current measurement mode or the voltage measurement mode based on the H₂O concentration acquired from the concentration calculating part 294, and as a result, detects the H₂O concentration by using either of the measurement modes. The H₂O concentration can be measured more accurately in a wide concentration range including the low concentration, by properly using the voltage measurement mode in which the measurement-object gas containing the low concentration of H₂O can be measured more accurately, and the current measurement mode in which the measurement-object gas containing the high concentration of H₂O can be measured more accurately.

In the step S13, when the measurement mode switching part 95 switches from the voltage measurement mode to the current measurement mode, substantially all of water vapor in the measurement-object gas is decomposed at the decomposing pump electrode 51 and substantially all of hydrogen generated by the decomposition is burned at the detection electrode 44, and therefore the atmosphere in the vicinity of the detection electrode 44 is controlled at a state where hydrogen derived from water vapor does not substantially exist. In this state, the measurement of the H₂O concentration in the current measurement mode (step S14) is performed at least once, and then the switching to the voltage measurement mode (step S23) is performed. Also, in the step S23, when the measurement mode switching part 95 switches from the current measurement mode to the voltage measurement mode, the pump current Ip2 is not applied to the current measurement pump cell 41 and the pump current Ip1 is applied to the decomposing pump cell 50 so that the voltage V2 detected in the electromotive force detection sensor cell 82 is at the set value V2_{SET}, thereby decomposing a part of water vapor in the measurement-object gas. Therefore, the atmosphere in the vicinity of the detection electrode 44 is controlled at a state where a predetermined amount of hydrogen derived from water vapor exists. In this state, the measurement of the H₂O concentration in the voltage measurement mode (step S24) is performed at least once, and then the switching to the current measurement mode (step S13) is performed.

As such, when the switching of the measurement mode is performed, the atmosphere in the vicinity of the detection electrode 44 is controlled so that the H₂O concentration can be measured, and then determination for the next switching of the measurement mode is performed. The switching of the measurement mode may usually be performed at intervals of one second or longer. The switching of the measurement mode is not intended to be the switching by turning on and off in such a minute time in which the above-described switching of the atmosphere in the vicinity of the detection electrode 44 cannot be realized, that is, the on and off control by so-called pulse current.

Immediately after the measurement mode switching part 95 switches from the voltage measurement mode to the current measurement mode in the step S13, the control of the decomposing pump cell 50 and the current measurement pump cell 41 is changed so that the pump current Ip2 may not be stable in some cases. Thus, the concentration calculating part 94 may perform the step S14 after a predetermined waiting time has elapsed. Also, immediately after the measurement mode switching part 95 switches from the current measurement mode to the voltage measurement mode in the step S23, the control of the decomposing pump cell 50 and the current measurement pump cell 41 is changed so that the voltage V1 may not be stable in some cases. Thus, the concentration calculating part 94 may perform the step S24 after a predetermined waiting time has elapsed.

The gas sensor 100 and the gas sensor 200 for detecting H₂O concentration in a measurement-object gas has been described above as examples of the embodiment according to the present invention, but the present invention is not limited thereto. The present invention may include a gas sensor having any structure including a sensor element and a control unit as long as the object of the present invention can be achieved, that is, the target gas to be measured is accurately measured in a wide concentration range including the low concentration of the target gas to be measured.

In the above embodiments, the gas sensor 100 and the gas sensor 200 detects the H₂O concentration in a measurement-object gas. However, the target gas to be measured is not limited to H₂O. For example, the target gas to be measured may be an oxide gas other than H₂O, such as carbon dioxide CO₂ and nitrogen oxide NOx, or a non-oxide gas such as ammonia NH₃.

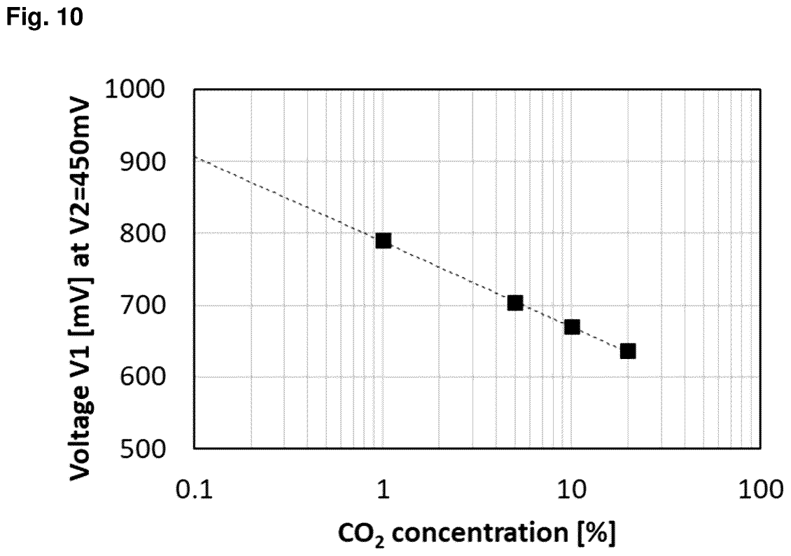

For example, the case of detecting carbon dioxide CO2 as a target gas to be measured will be described. When carbon dioxide CO₂ is decomposed (or, reduced), carbon monoxide CO and oxygen O₂ are generated (2CO₂→2CO+O₂). Carbon monoxide CO is a reducing gas as well as hydrogen H₂. Therefore, in the case of detecting carbon dioxide CO₂, it is possible to calculate a CO₂ concentration in a measurement-object gas on the basis of the voltage V1 between the decomposing pump electrode 51 and the reference electrode 42, as in the case of the gas sensor 100. FIG. 10 is a graph showing one example of a relationship between CO₂ concentration and the voltage V1. The horizontal axis of the graph represents the CO₂ concentration (%) and the vertical axis of the graph represents the voltage V1 (mV) when the voltage V2 is at 450 mV. Thus, a correlation can be obtained between the CO₂ concentration and the voltage V1, similar to that between H₂O concentration and voltage V1 shown in FIG. 5. The concentration calculating part may calculate the CO₂ concentration in the measurement-object gas on the basis of the voltage V1 by using such a correlation shown in FIG. 10. Further, in the same manner as the gas sensor 200 described above, the measurement can be performed while switching between the voltage measurement mode and the current measurement mode.

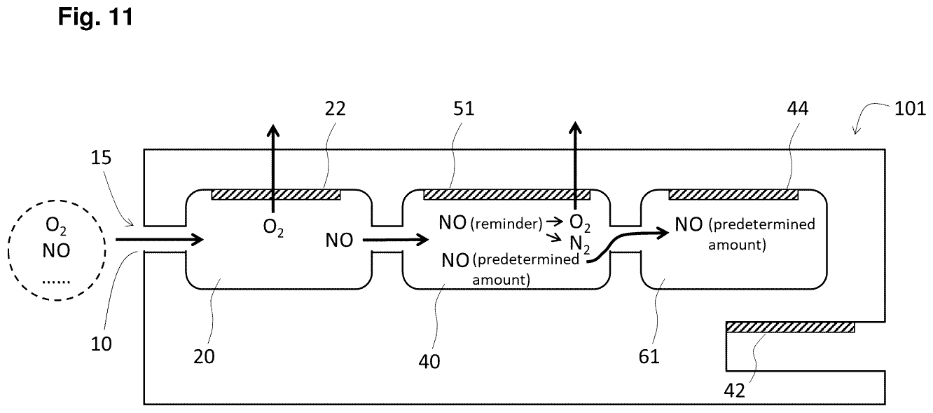

For example, the case of detecting nitrogen oxide NOx as a target gas to be measured will be described referring to FIG. 1 and FIG. 11. FIG. 11 is a conceptual diagram showing a principle of measuring nitrogen oxide, in the case of measuring nitrogen oxide by using the gas sensor 100. In FIG. 11, only nitrogen oxide NOx and oxygen O₂ are shown as gas components in the measurement-object gas. When nitrogen oxide NOx is decomposed (or, reduced), nitrogen N₂ and oxygen O₂ are generated (2NO→N₂+O₂). Nitrogen N₂ is a chemically stable inert gas. In case of detecting nitrogen oxide NOx, the voltage V2 between the detection electrode 44 and the reference electrode 42 is generated in accordance with NOx that is not decomposed at the decomposing pump electrode 51. Therefore, control is performed so that a predetermined amount of NOx is not decomposed at the decomposing pump electrode 51 regardless of NOx concentration in the measurement-object gas. That is, when NOx concentration in the measurement-object gas is high, NOx may be decomposed at a high decomposition rate at the decomposing pump electrode 51. On the other hand, when NOx concentration in the measurement-object gas is low, water vapor may be decomposed at a low decomposition rate at the decomposing pump electrode 51. As a result, a predetermined amount of NOx remains without being decomposed, regardless of NOx concentration in the measurement-object gas. Then, the measurement-object gas containing the predetermined amount of NOx that is not decomposed is introduced into the third internal cavity 61 and reaches the detection electrode 44. Therefore, unlike the case of H₂O or CO₂, it is considered that the higher the NOx concentration in the measurement-object gas, the higher the voltage V1 tends to be. The concentration calculating part can calculate a NOx concentration in the measurement-object gas based on the voltage V1 by using a correlation between the NOx concentration and the voltage V1, as in the case of the gas sensor 100. Further, the measurement can be performed while switching between the voltage measurement mode and the current measurement mode. In the current measurement mode, it is preferable that NOx is not substantially decomposed at the decomposing pump electrode 51 and substantially all of NOx is decomposed at the detection electrode 44. For example, the pump current Ip1 may not be applied to the decomposing pump cell 50 in the current measurement mode.

For example, the case where ammonia NH₃ is detected as a target gas to be measured will be described. In the case of detecting ammonia NH₃, ammonia NH₃ is decomposed and converted to nitrogen oxide NO at the intracavity oxygen pump electrode (namely, the inner main pump electrode 22) of the oxygen pump cell (namely, the main pump cell 21). It is possible to measure a concentration of ammonia NH₃ by measuring the NO converted from ammonia NH₃ in the same manner as for measuring NOx described above.

In the case of measuring a H₂O concentration or a CO2 concentration, for example, the set value V0_{SET} used for controlling the main pump cell 21 may be set to about 300 mV to 800 mV. More preferably, the set value V0_{SET} may be set to about 350 mV to 500 mV. H₂O and CO₂ are not to be substantially decomposed in the main pump cell 21. It is to be noted that NOx may be decomposed in the main pump cell 21 when NOx is present in the measurement-object gas. In this case, the inner main pump electrode 22 may contain, for example, Rh as a metal component. In the case of detecting a NOx concentration or a NH₃ concentration, for example, the set value V0_{SET} used for controlling the main pump cell 21 may be set to about 150 mV to 450 mV. NOx is not to be substantially decomposed in the main pump cell 21. Accordingly, the set value V0_{SET} used for controlling the main pump cell 21 may appropriately be set depending on a kind of the target gas to be measured.

In the case of performing feedback control for adjusting the voltage V1 in the oxygen-partial-pressure detection sensor cell 81 for decomposing pump control so that the voltage V2 between the detection electrode 44 and the reference electrode 42 in the electromotive force detection sensor cell 82 is at the set value V2_{SET}, the set value V2_{SET} may appropriately be set depending on a kind of the target gas to be measured. When a CO₂ concentration is measured, the set value V2_{SET} may be set to about 300 mV to 600 mV as in the case of measuring H₂O concentration. More preferably, the set value V2_{SET} may be set to about 400 mV to 500 mV. In the case of detecting a NOx concentration or a NH₃ concentration, the set value V2_{SET} may be set to, for example, about 300 mV to 600 mV. More preferably, the set value V2_{SET} may be set to about 400 mV to 500 mV.

In the current measurement mode, the set value V1a_{SET} used for controlling the decomposing pump cell 50, and the set value V2a_{SET} used for controlling the current measurement pump cell 41 may appropriately be set depending on a kind of the target gas to be measured.

By setting each of the set values appropriately as described above, it may be possible to reduce the influence of other gasses other than a desired target gas to be measured and measure the desired target gas to be measured more accurately.

In the above embodiment, the pump control part 93 of the control part 91 performs feedback control for adjusting the voltage V1 in the oxygen-partial-pressure detection sensor cell 81 for decomposing pump control so that the voltage V2 between the detection electrode 44 and the reference electrode 42 in the electromotive force detection sensor cell 82 is at the set value V2_{SET}. However, the control method is not limited thereto. For example, the pump control part 93 may perform feedback control to the pump voltage Vp1 of the variable power supply 52 in the decomposing pump cell 50 so that the voltage V2 between the detection electrode 44 and the reference electrode 42 in the electromotive force detection sensor cell 82 is at the set value V2_{SET}. Then, the concentration calculating part 94 may calculate a concentration of the target gas to be measured in the measurement-object gas is calculated based on the voltage V1 between the decomposing pump electrode 51 and the reference electrode 42 in the oxygen-partial-pressure detection sensor cell 81 for decomposing pump control detected as a result of the control.

In the gas sensor 100 of the above embodiment, as shown in FIG. 1, the sensor element 101 has a structure in which three internal cavities, the first internal cavity 20, the second internal cavity 40, and the third internal cavity 61 are provided and the inner main pump electrode 22, the decomposing pump electrode 51, and the detection electrode 44 are respectively disposed in these internal cavities. However, the structure of the sensor element 101 is not limited thereto. For example, the sensor element 101 may have a structure in which two internal cavities, the first internal cavity 20 and the second internal cavity 40 are provided, the inner main pump electrode 22 is disposed in the first internal cavity 20, and the decomposing pump electrode 51 and the detection electrode 44 are disposed in the second internal cavity 40. In this case, for example, a porous protective layer covering the detection electrode 44 may be formed as a diffusion-rate limiting part between the decomposing pump electrode 51 and the detection electrode 44.

In the gas sensor 100 and the gas sensor 200 of the above embodiments, the outer pump electrode 23 has two or three functions as an extracavity oxygen pump electrode in the oxygen pump cell (namely, the main pump cell 21), an extracavity decomposing pump electrode in the decomposing pump cell 50, and, in case of the gas sensor 200, an extracavity detection electrode in the current measurement pump cell 41. However, the outer pump electrode 23 is not limited thereto. For example, the extracavity decomposing pump electrode, the extracavity decomposing pump electrode, and the extracavity detection electrode may be formed as different electrodes. For example, any one or more of the extracavity decomposing pump electrode, the extracavity decomposing pump electrode, and the extracavity detection electrode may be provided on the outer surface of the base part 102 separately from the outer pump electrode 23 so as to be in contact with a measurement-object gas. Alternatively, the reference electrode 42 may also serve as any one or more of the extracavity decomposing pump electrode, the extracavity decomposing pump electrode, and the extracavity detection electrode.

In the above gas sensor 200, a switch is provided as one example of the switching unit 47, but the present invention is not limited thereto. For example, the variable power supply 46 may be used as the switching unit 47. The measurement mode switching part 95 may set the pump voltage Vp2 in the variable power supply 46 to zero not to apply a voltage in the current measurement pump cell 41 so that a current does not flow through the current measurement pump cell 41 in case of switching to the voltage measurement mode, and may set the pump voltage Vp2 in the variable power supply 46 to a predetermined value to apply the predetermined voltage in the current measurement pump cell 41 so that a current flows through the current measurement pump cell 41 in case of switching to the current measurement mode. It is to be noted that, in the current measurement mode, the feedback control may be performed to the pump voltage Vp2 of the variable power supply 46 in the current measurement pump cell 41 so that the electromotive force V2 as the control voltage detected in the electromotive force detection sensor cell 82 is at the set value V2_{SET}, as in the case of the above gas sensor 200.

In the above gas sensor 200, the measurement mode switching part 95 first switches to the current measurement mode in the step S13 after the step S12, but the measurement mode switching part 95 may first switch to the voltage measurement mode in the step S23 after the step S12.

Alternatively, at a startup of the gas sensor 200, the control unit 290 may be preset in the current measurement mode, or may be preset in the voltage measurement mode.

In the above gas sensor 200, the measurement mode switching part 95 switches between the voltage measurement mode and the current measurement mode based on the H₂O concentration calculated by the concentration calculating part 294. However, the present invention is not limited thereto.

As a threshold value for switching from the current measurement mode to the voltage measurement mode, instead of the first concentration threshold value C1, a lower limit of the pump current Ip2 at which a desired measurement accuracy can be obtained when measuring in the current measurement mode may appropriately be set by a person skilled in the art. As a threshold value for switching from the voltage measurement mode to the current measurement mode, instead of the second concentration threshold value C2, an upper limit of the voltage V1 at which a desired measurement accuracy can be obtained when measuring in the voltage measurement mode may appropriately be set by a person skilled in the art.

The switching between the voltage measurement mode and the current measurement mode may be performed, for example, based on a signal sent from other devices such as the ECU and the exhaust gas treatment system of the car.

As described above, according to the present invention, measurement is performed based on a voltage between the intracavity decomposing pump electrode and the reference electrode when a voltage between the intracavity detection electrode and the reference electrode is at a predetermined value, so that it is possible to accurately measure the target gas to be measured in a wide concentration range including the low concentration of the target gas to be measured. Further, measurement may be performed while switching between the voltage measurement mode in which the measurement accuracy in low concentration is higher, and the current measurement mode in which the measurement accuracy in high concentration is higher, so that it is possible to more accurately measure the target gas to be measured in a wide concentration range including the low concentration of the target gas to be measured. It is possible to accurately measure in a wide concentration range of, for example, 0.01% to 30% in case of measuring water vapor or carbon dioxide, or in a wide concentration range of, for example, 10 ppm to 5000 ppm in case of measuring NOx or NH₃.

### EXPLANATION OF REFERENCE SIGNS IN THE DRAWINGS

1: first substrate layer; 2: second substrate layer; 3: third substrate layer; 4: first solid electrolyte layer; 5: spacer layer; 6: second solid electrolyte layer; 10: gas inlet; 11: first diffusion-rate limiting part; 12: buffer space; 13: second diffusion-rate limiting part; 15: measurement-object gas flow cavity; 20: first internal cavity; 21: main pump cell; 22: inner main pump electrode; 22a: ceiling electrode portion (of the inner main pump electrode); 22b: bottom electrode portion (of the inner main pump electrode); 23: outer pump electrode; 24: variable power supply (of the main pump cell); 30: third diffusion-rate limiting part; 40: second internal cavity; 41: current measurement pump cell; 42: reference electrode; 43: reference gas introduction space; 44: detection electrode; 46: variable power supply (of the current measurement pump cell); 47: switching unit; 48: air introduction layer; 50: decomposing pump cell; 51: decomposing pump electrode; 51a: ceiling electrode portion (of the decomposing pump electrode); 51b: bottom electrode portion (of the decomposing pump electrode); 52: variable power supply (of the decomposing pump cell); 60: fourth diffusion-rate limiting part; 61: third internal cavity; 70: heater part; 71: heater electrode; 72: heater; 73 through hole; 74 heater insulating layer; 75: pressure relief vent; 76: heater lead; 77: heater power supply; 80: oxygen-partial-pressure detection sensor cell for main pump control; 81: oxygen-partial-pressure detection sensor cell for decomposing pump control; 82: electromotive force detection sensor cell; 83: sensor cell; 85: feed back switching part; 90, 290: control unit; 91, 291: control part; 92: heater control part; 93, 293: pump control part; 94, 294: concentration calculating part; 95: measurement mode switching part; 100, 200: gas sensor; 101, 201: sensor element; and 102: base part.

What is claimed is:

1. A gas sensor for detecting a target gas to be measured in a measurement-object gas, the gas sensor comprising a sensor element and a control unit for controlling the sensor element, wherein
the sensor element comprises:
a base part in an elongated plate shape, including an oxygen-ion-conductive solid electrolyte layer;
a measurement-object gas flow cavity formed from one end part in a longitudinal direction of the base part;
an oxygen pump cell for adjusting an oxygen concentration in a measurement-object gas, the oxygen pump cell including: an intracavity oxygen pump electrode disposed in the measurement-object gas flow cavity; and an extracavity oxygen pump electrode disposed at a position different from the measurement-object gas flow cavity on the base part and corresponding to the intracavity oxygen pump electrode;
a decomposing pump cell for decomposing a target gas to be measured in the measurement-object gas, the decomposing pump cell including: an intracavity decomposing pump electrode disposed at a position farther from the one end part in the longitudinal direction of the base part than the intracavity oxygen pump electrode in the measurement-object gas flow cavity; and an extracavity decomposing pump electrode disposed at a position different from the measurement-object gas flow cavity on the base part and corresponding to the intracavity decomposing pump electrode;
an intracavity detection electrode disposed at a position farther from the one end part in the longitudinal direction of the base part than the intracavity decomposing pump electrode in the measurement-object gas flow cavity; and
a reference electrode disposed inside the base part to be in contact with a reference gas; and
the control unit comprises:
a pump control part for controlling operation of the oxygen pump cell and the decomposing pump cell, and
a concentration calculating part for calculating a concentration of the target gas to be measured in the measurement-object gas, wherein
the pump control part operates the oxygen pump cell to adjust an oxygen concentration in the measurement-object gas to a predetermined concentration,
the pump control part operates the decomposing pump cell so that a voltage between the intracavity detection electrode and the reference electrode is at a predetermined value, thereby decomposing at least a part of the target gas to be measured in the measurement-object gas at the intracavity decomposing pump electrode of the decomposing pump cell and pumping out, by the decomposing pump cell, oxygen generated by decomposing the target gas to be measured, and
the concentration calculating part calculates a concentration of the target gas to be measured in the measurement-object gas based on a value of a voltage between the intracavity decomposing pump electrode and the reference electrode.

2. The gas sensor according to claim 1, wherein
the pump control part adjusts the voltage between the intracavity decomposing pump electrode and the reference electrode so that the voltage between the intracavity detection electrode and the reference electrode is at the predetermined value, thereby decomposing at least the part of the target gas to be measured in the measurement-object gas at the intracavity decomposing pump electrode of the decomposing pump cell and pumping out, by the decomposing pump cell, oxygen generated by decomposing the target gas to be measured, and
the concentration calculating part calculates the concentration of the target gas to be measured in the measurement-object gas based on a value of the adjusted voltage between the intracavity decomposing pump electrode and the reference electrode.

3. The gas sensor according to claim 1, wherein
the sensor element further comprises a current measurement pump cell including the intracavity detection electrode, and an extracavity detection electrode disposed at a position different from the measurement-object gas flow cavity on the base part and corresponding to the intracavity detection electrode; and
the control unit further comprises a switching unit for switching whether a current flows through the current measurement pump cell or not.

4. The gas sensor according to claim 3, wherein the control unit comprises:
a measurement mode switching part for switching between a voltage measurement mode in which a concentration of the target gas to be measured in the measurement-object gas is calculated based on the value of the voltage between the intracavity decomposing pump electrode and the reference electrode, and a current measurement mode in which a concentration of the target gas to be measured in the measurement-object gas is calculated based on a current value flowing through the current measurement pump cell, and
the measurement mode switching part switches the switching unit so that a current does not flow through the current measurement pump cell in case of switching to the voltage measurement mode, and switches the switching unit so that a current flows through the current measurement pump cell in case of switching to the current measurement mode.

5. The gas sensor according to claim 4, wherein
the measurement mode switching part switches to the voltage measurement mode when the measurement mode switching part determines that a concentration of the target gas to be measured detected in the current measurement mode is lower than a predetermined first concentration threshold value C1, and
the measurement mode switching part switches to the current measurement mode when the measurement mode switching part determines that a concentration of the target gas to be measured detected in the voltage measurement mode is higher than a predetermined second concentration threshold value C2.

6. The gas sensor according to claim 5, wherein the first concentration threshold value C1 is lower than the second concentration threshold value C2.

7. The gas sensor according to claim 4, wherein
in the current measurement mode, a current value in the current measurement pump cell is controlled so that a voltage between the intracavity detection electrode and the reference electrode is at a predetermined value.

8. The gas sensor according to claim 1, wherein the target gas to be measured is selected from the group consisting of water vapor H₂O, carbon dioxide CO₂, and nitrogen oxide NOx.

9. A control method of a gas sensor for detecting a target gas to be measured in a measurement-object gas, the gas sensor comprising a sensor element and a control unit for controlling the sensor element, wherein
the sensor element comprises:
a base part in an elongated plate shape, including an oxygen-ion-conductive solid electrolyte layer;
a measurement-object gas flow cavity formed from one end part in a longitudinal direction of the base part;
an oxygen pump cell for adjusting an oxygen concentration in a measurement-object gas, the oxygen pump cell including: an intracavity oxygen pump electrode disposed in the measurement-object gas flow cavity; and an extracavity oxygen pump electrode disposed at a position different from the measurement-object gas flow cavity on the base part and corresponding to the intracavity oxygen pump electrode;
a decomposing pump cell for decomposing a target gas to be measured in the measurement-object gas, the decomposing pump cell including: an intracavity decomposing pump electrode disposed at a position farther from the one end part in the longitudinal direction of the base part than the intracavity oxygen pump electrode in the measurement-object gas flow cavity; and an extracavity decomposing pump electrode disposed at a position different from the measurement-object gas flow cavity on the base part and corresponding to the intracavity decomposing pump electrode;
an intracavity detection electrode disposed at a position farther from the one end part in the longitudinal direction of the base part than the intracavity decomposing pump electrode in the measurement-object gas flow cavity; and
a reference electrode disposed inside the base part to be in contact with a reference gas; and
the control unit comprises:
a pump control part for controlling operation of the oxygen pump cell and the decomposing pump cell, and
a concentration calculating part for calculating a concentration of the target gas to be measured in the measurement-object gas, and
the control method comprising:
a pump controlling step of operating the oxygen pump cell by the pump control part to adjust an oxygen concentration in the measurement-object gas to a predetermined concentration, and
operating the decomposing pump cell so that a voltage between the intracavity detection electrode and the reference electrode is at a predetermined value by the pump control part, thereby decomposing at least a part of the target gas to be measured in the measurement-object gas at the intracavity decomposing pump electrode of the decomposing pump cell and pumping out, by the decomposing pump cell, oxygen generated by decomposing the target gas to be measured, and
a concentration calculating step of calculating a concentration of the target gas to be measured in the measurement-object gas based on a value of a voltage between the intracavity decomposing pump electrode and the reference electrode by the concentration calculating part.
